(19) United States
(12) Patent Application Publication
(10) Pub. No.: US 2003/0148339 A1
(43) Pub. Date: Aug. 7, 2003

(54) Artificial genes for use as controls in gene expression analysis systems

(76) Inventors: Hrissi Samartzidou, Phoenix, AZ (US); Thomas Houts, Gilroy, CA (US); Wen Yang, Palo Alto, CA (US); Son Bui, Milpitas, CA (US); Timothy Harkins, San Jose, CA (US)

Correspondence Address: Amersham Biosciences Corp., 800 Centennial Avenue, Piscataway, NJ, 08855, US

(21) Appl. No.: 10/278,845

(22) Filed: Oct. 23, 2002

Related U.S. Application Data
(63) Continuation-in-part of application No. 10/140,545, filed on May 7, 2002.
(60) Provisional application No. 60/289,202, filed on May 7, 2001.
(60) Provisional application No. 60/312,420, filed on Aug. 15, 2001.
(60) Provisional application No. 60/335,115, filed on Oct. 24, 2001.
(60) Provisional application No. 60/391,367, filed on Jun. 25, 2002.

(51) Int. Cl. C12Q 1/68; C07H 21/04

(52) U.S. Cl. 435/6; 536/24.3

(57) ABSTRACT

Method of producing universal controls for use in gene expression analysis systems such as macroarrays, real-time PCR, northern blots, SAGE and microarrays. The controls are generated either from near-random sequence of DNA, or from intergenic or intronic regions of a genome. Twenty-three specific control sequences are also disclosed. Also presented are methods of using these controls, including as negative controls, positive controls, and as calibrators of a gene expression analysis system.

### CROSS-REFERENCE TO RELATED APPLICATIONS

This application is a continuation-in-part of U.S. patent application Ser. No. 10/140,545, filed May 7, 2002, which claims priority to U.S. provisional patent application Nos. 60/289,202, filed May 7, 2001, and 60/312,420, filed Aug. 15, 2001. This application also claims priority to U.S. provisional patent application Ser. Nos. 60/335,115, filed Oct. 24, 2001, and 60/391,367, filed Jun. 25, 2002, the disclosures of which are incorporated herein by reference in their entireties.

### REFERENCE TO SEQUENCE LISTING SUBMITTED ON COMPACT DISC

The present application includes a Sequence Listing filed on one CD-R disc, provided in duplicate, containing a single file named pto_PB0181.txt, having 56 kilobytes, last modified on Oct. 21, 2002, and recorded on Oct. 21, 2002. The Sequence Listing contained in said file on said disc is incorporated herein by reference in its entirety.

### BACKGROUND OF THE INVENTION

1. Field of the Invention

The present invention relates to a method of using artificial genes as universal controls in gene expression analysis systems. More particularly, the present invention relates to a method of producing universal Controls for use in gene expression analysis systems such as macroarrays, real-time PCR, northern blots, SAGE and microarrays, such as those provided in the Microarray ScoreCard system.

2. Description of Related Art

Gene expression profiling is an important biological approach used to better understand the molecular mechanisms that govern cellular function and growth. Microarray analysis is one of the tools that can be applied to measure the relative expression levels of individual genes under different conditions. Microarray measurements often appear to be systematically biased, however, and the factors that contribute to this bias are many and ill-defined (Bowtell, D. L., Nature Genetics 21, 25-32 (1999); Brown, P. P. and Botstein, D., Nature Genetics 21, 33-37 (1999)). Others have recommended the use of “spikes” of purified mRNA at known concentrations as controls in microarray experiments. Affymetrix includes several for use with their GeneChip products. In the current state of the art, these selected genes are actual genes selected from very distantly related organisms. For example, the human chip (designed for use with human mRNA) includes control genes from bacterial and plant sources.

Each of the prior art controls consists of transcribed sequences of DNA from some source. As a result, that source cannot be the subject of a hybridization experiment using those controls due to the inherent hybridization of the controls to its source. In addition, the lack of universal references consistent from experiment to experiment and from species to species greatly reduces the ability for scientists to compare data across labs, users, or time. What is needed, therefore, is a set of universal controls that do not hybridize with the DNA of any source which may be the subject of an experiment. More desirably, there is a need for a universal control for gene expression analysis which do not hybridize with any known source.

### SUMMARY OF THE INVENTION

Accordingly, this invention provides a process of producing universal controls that are useful in gene expression analysis systems designed for any species and which can be tested to insure lack of hybridization with mRNA from sources other than the control DNA itself.

The invention relates in a first embodiment to a process for producing at least one universal control for use in a gene expression analysis system. The process comprises selecting at least one non-transcribed (preferably intergenic, also intronic) region of genomic DNA from a known sequence, designing primer pairs for said at least one non-transcribed region and amplifying said at least one non-transcribed region of genomic DNA to generate corresponding double stranded DNA, then cloning said double stranded DNA using a vector to obtain additional double stranded DNA and formulating at least one control comprising said double stranded DNA.

The present invention relates in a second embodiment to a process of producing at least one universal control for use in a gene expression analysis system wherein testing of said at least one non-transcribed region to ensure lack of hybridization with mRNA from sources other than said at least one non-transcribed region of genomic DNA is performed.

The present invention in a third embodiment relates to said process further comprising purifying said DNA and mRNA, determining the concentrations thereof and formulating at least one control comprising said DNA or of said mRNA at selected concentrations and ratios.

Another embodiment of the present invention is a universal control for use in a gene expression analysis system comprising a known amount of at least one DNA generated from at least one non-transcribed region of genomic DNA from a known sequence, or comprising a known amount of at least one mRNA generated from DNA generated from at least one non-transcribed region of genomic DNA from a known sequence. The present invention may optionally include generating mRNA complementary to said DNA and formulating at least one control comprising said mRNA, by optionally purifying said DNA and mRNA, determining the concentrations thereof and formulating at least one control comprising said DNA or of said mRNA at selected concentrations and ratios.

Another embodiment of the present invention is a universal control for use in a gene expression analysis system wherein a known amount of at least one DNA sequence generated from at least one non-transcribed region of genomic DNA from a known sequence, a known amount of at least one mRNA generated from DNA generated from at least one non-transcribed region of genomic DNA from a known sequence is included, and the aforementioned control wherein, said DNA and mRNA do not hybridize with any DNA or mRNA from a source other than the at least one non-transcribed region of genomic DNA.

The present invention, relates to a method of using said universal control, as a negative control in a gene expression analysis system by adding a known amount of said control containing a known amount of DNA, to a gene expression analysis system as a control sample and subjecting the sample to hybridization conditions in the absence of complementary labeled mRNA and examining the control sample for the absence or presence of signal.

Further, said controls can be used in a gene expression analysis system by adding a known amount of a said control containing a known amount of DNA to a gene expression analysis system as a control sample and subjecting the sample to hybridization conditions, in the presence of a said control containing a known amount of labeled complementary mRNA, and measuring the signal values for the labeled mRNA and determining the expression level of the gene transcript based on the signal value of the labeled mRNA.

Additionally, said controls may be used as calibrators in a gene expression analysis system by adding a known amount of a said control containing known amounts of several DNA sequences to a gene expression analysis system as control samples and subjecting the samples to hybridization conditions in the presence of a said control containing known amounts of corresponding complementary labeled mRNAs, each mRNA being at a different concentration and measuring the signal values for the labeled mRNAs and constructing a dose-response or calibration curve based on the relationship between signal value and concentration of each mRNA.

Also, the present invention relates to a method of using said controls as calibrators for gene expression ratios in a two-color gene expression analysis system by adding a known amount of at least one of said controls containing a known amount of DNA to a two-color gene expression analysis system as control samples and subjecting the samples to hybridization conditions in the presence of a said control containing known amounts of two differently labeled corresponding complementary labeled mRNAs for each DNA sample present and measuring the ratio of the signal values for the two differently labeled mRNAs and comparing the signal ratio to the ratio of concentrations of the two or more differently labeled mRNAs.

A further embodiment of the present invention is a process of producing controls that are useful in gene expression analysis systems designed for any species and which can be tested to insure lack of hybridization with mRNA from sources other than the synthetic sequences of DNA from which the control is produced.

One or more such controls can be produced by a process comprising synthesizing a near-random sequence of non-transcribed DNA, designing primer pairs for said at least one near random sequence and amplifying said non-transcribed DNA to generate corresponding double stranded DNA, then cloning said double stranded DNA using a vector to obtain additional double stranded DNA and formulating at least one control comprising said double stranded DNA.

The process can also be used to produce at least one control for use in a gene expression analysis system wherein testing of said sequence of non-transcribed synthetic DNA to ensure lack of hybridization with mRNA from sources other than said sequence of non-transcribed DNA is performed.

Additionally, mRNA complementary to said synthetic DNA can be generated and formulated to generate at least one control comprising said mRNA.

DNA and mRNA can be subsequently purified, the concentrations thereof determined, and one or more controls comprising said DNA or said mRNA at selected concentrations and ratios be formulated.

Another embodiment of the present invention is a control for use in a gene expression analysis system produced by the process comprises synthesizing a near-random sequence of DNA, designing primer pairs for said synthetic DNA and amplifying said DNA to generate corresponding double stranded DNA, then cloning said double stranded DNA using a vector to obtain additional double stranded DNA and formulating at least one control comprising a known amount of at least one said double stranded DNA or a known amount of at least one mRNA generated from said DNA, and optionally, wherein, said DNA and mRNA do not hybridize with any DNA or mRNA from a source other than said DNA sequence of non-transcribed DNA.

The present invention, additionally, relates to a method of using said controls containing a known amount of DNA, as a negative control in a gene expression analysis system including adding a known amount of said control containing a known amount of DNA to a gene expression analysis system as a control sample, and subjecting the sample to hybridization conditions in the absence of complementary labeled mRNA and examining the control sample for the absence or presence of signal.

Further, said controls may be used in a gene expression analysis system wherein a known amount of a said control containing a known amount of DNA is added to a gene expression analysis system as a control sample and subjecting the sample to hybridization conditions in the presence of a said control containing a known amount of labeled complementary mRNA and measuring the signal values for the labeled mRNA and determining the expression level of the gene transcript based on the signal value of the labeled mRNA.

The present invention, also relates to a method of using said controls as calibrators in a gene expression analysis system including adding known amounts of a said control containing known amounts of several DNAs to a gene expression analysis system as control samples and subjecting the samples to hybridization conditions in the presence of a said control containing known amounts of corresponding complementary labeled mRNAs, each mRNA being at a different concentration and measuring the signal values for the labeled mRNAs and constructing a dose-response or calibration curve based on the relationship between signal value and concentration of each mRNA.

The present invention, additionally, relates to a method of using said controls as calibrators for gene expression ratios in a two-color gene expression analysis system comprising adding a known amount of at least one of said controls containing a known amount of DNA to a two-color gene expression analysis system as control samples and subjecting the samples to hybridization conditions in the presence of a said control containing known amounts of two differently labeled corresponding complementary labeled mRNAs for each DNA sample present and measuring the ratio of the signal values for the two differently labeled mRNAs and comparing the signal ratio to the ratio of concentrations of the two or more differently labeled mRNAs.

Further embodiments and uses of the current invention will become apparent from a consideration of the ensuing description.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1 shows representative results for the selection of universal controls that do not cross-hybridize with human RNA;

FIG. 2 shows representative results for the selection of universal controls that do not cross-hybridization with each other;

FIG. 3 represents a performance evaluation of the universal controls;

FIG. 4 shows a scatter plot of raw signals for the calibration and ratio controls from a two-color hybridization experiment;

FIG. 5 shows calibration curves based on the Calibration controls for a representative hybridization experiment;

FIG. 6 presents the control nucleotide sequence of DR1 (SEQ ID NO: 1);

FIG. 7 presents the control nucleotide sequence of DR2 (SEQ ID NO: 2);

FIG. 8 presents the control nucleotide sequence of DR3 (SEQ ID NO: 3);

FIG. 9 presents the control nucleotide sequence of DR4 (SEQ ID NO: 4);

FIG. 10 presents the control nucleotide sequence of DR5 (SEQ ID NO: 5);

FIG. 11 presents the control nucleotide sequence of DR6 (SEQ ID NO: 6);

FIG. 12 presents the control nucleotide sequence of DR7 (SEQ ID NO: 7);

FIG. 13 presents the control nucleotide sequence of DR8 (SEQ ID NO: 8);

FIG. 14 presents the control nucleotide sequence of DR9 (SEQ ID NO: 9);

FIG. 15 presents the control nucleotide sequence of DR10 (SEQ ID NO: 10);

FIG. 16 presents the control nucleotide sequence of RC1 (SEQ ID NO: 11);

FIG. 17 presents the control nucleotide sequence of RC2 (SEQ ID NO: 12);

FIG. 18 presents the control nucleotide sequence of RC3 (SEQ ID NO: 13);

FIG. 19 presents the control nucleotide sequence of RC4 (SEQ ID NO: 14);

FIG. 20 presents the control nucleotide sequence of RC5 (SEQ ID NO: 15);

FIG. 21 presents the control nucleotide sequence of RC6 (SEQ ID NO: 16);

FIG. 22 presents the control nucleotide sequence of RC7 (SEQ ID NO: 17);

FIG. 23 presents the control nucleotide sequence of RC8 (SEQ ID NO: 18);

FIG. 24 presents the control nucleotide sequence of Utility1 (SEQ ID NO: 19);

FIG. 25 presents the control nucleotide sequence of Utility2 (SEQ ID NO: 20);

FIG. 26 presents the control nucleotide sequence of Utility3 (SEQ ID NO: 21);

FIG. 27 presents the control nucleotide sequence of Negative1 (SEQ ID NO: 22);

FIG. 28 presents the control nucleotide sequence of Negative2 (SEQ ID NO: 23);

FIG. 29 presents the nucleotide sequence of DR1s used in a spike mix (SEQ ID NO: 24);

FIG. 30 presents the nucleotide sequence of DR2s used in a spike mix (SEQ ID NO: 25);

FIG. 31 presents the nucleotide sequence of DR3s used in a spike mix (SEQ ID NO: 26);

FIG. 32 presents the nucleotide sequence of DR4s used in a spike mix (SEQ ID NO: 27);

FIG. 33 presents the nucleotide sequence of DR5s used in a spike mix (SEQ ID NO: 28);

FIG. 34 presents the nucleotide sequence of DR6s used in a spike mix (SEQ ID NO: 29);

FIG. 35 presents the nucleotide sequence of DR7s used in a spike mix (SEQ ID NO: 30);

FIG. 36 presents the nucleotide sequence of DR8s used in a spike mix (SEQ ID NO: 31);

FIG. 37 presents the nucleotide sequence of DR9s used in a spike mix (SEQ ID NO: 32);

FIG. 38 presents the nucleotide sequence of DR10s used in a spike mix (SEQ ID NO: 33);

FIG. 39 presents the nucleotide sequence of RC1s used in a spike mix (SEQ ID NO: 34);

FIG. 40 presents the nucleotide sequence of RC2s used in a spike mix (SEQ ID NO: 35);

FIG. 41 presents the nucleotide sequence of RC3s used in a spike mix (SEQ ID NO: 36);

FIG. 42 presents the nucleotide sequence of RC4s used in a spike mix (SEQ ID NO: 37);

FIG. 43 presents the nucleotide sequence of RC5s used in a spike mix (SEQ ID NO: 38);

FIG. 44 presents the nucleotide sequence of RC6s used in a spike mix (SEQ ID NO: 39);

FIG. 45 presents the nucleotide sequence of RC7s used in a spike mix (SEQ ID NO: 40);

FIG. 46 presents the nucleotide sequence of RC8s used in a spike mix (SEQ ID NO: 41);

FIG. 47 presents the nucleotide sequence of Utility1s used in a spike mix (SEQ ID NO: 42);

FIG. 48 presents the nucleotide sequence of Utility2s used in a spike mix (SEQ ID NO: 43);

FIG. 49 presents the nucleotide sequence of Utility3s used in a spike mix (SEQ ID NO: 44);

FIG. 50 presents the nucleotide sequence of Negative1s used in a spike mix (SEQ ID NO: 45); and

FIG. 51 presents the nucleotide sequence of Negative2s used in a spike mix (SEQ ID NO: 46).

### DETAILED DESCRIPTION OF THE INVENTION

The present invention teaches universal Controls for use in gene expression analysis systems such as microarrays. Many have expressed interest in being able to obtain suitable genes and spikes as controls for inclusion in their arrays.

An advantage of the universal Controls of this invention is that a single set can be used with assay systems designed for any species, as these Controls will not be present unless intentionally added. This contrasts with the concept of using genes from “distantly related species.” For example, an analysis system directed at detecting human gene expression might employ a Bacillus subtilis gene as control, which may not be present in a human genetic material. But this control might be present in bacterial genetic material (or at least, cross hybridize), thus it may not be a good control for an experiment on bacterial gene expression. The novel universal Controls presented here provide an advantage over the state of the art in that the same set of controls can be used without regard to the species for the test sample RNA.

The present invention employs the novel approaches of using either non-transcribed genomic sequences or totally random synthetic sequences as a template and generating both DNA and complementary “mRNA” from such sequences, for use as controls. The Controls could be devised de novo by designing near-random sequences and synthesizing them resulting in synthetic macromolecules as universal controls. Totally synthetic random DNA fragments are so designed that they do not cross-hybridize with each other or with RNA from any biologically relevant species (meaning species whose DNA or RNA might be present in the gene expression analysis system). The cost of generating such large synthetic DNA molecules can be high. However, they only need to be generated a single time. Additionally, fragment size can be increased by ligating smaller synthetic fragments together by known methods. In this way, fragments large enough to be easily cloned can be created. Through cloning and PCR sufficient quantities of DNA for use as controls can be produced and mRNA can be generated by in vitro transcription for use in controls.

A simpler approach is to identify sequences from the intergenic or intronic regions (referred here as non-transcribed regions) of genomic DNA from an organism, and use these as a template for synthesis via PCR (polymerase chain reaction). Ideally, sequences of around 1000 bases (could range from 500 to 2000 bases) are selected based on computer searches of publicly accessible sequence data. The criteria for selection include:

1. The sequence must be from a non-transcribed region; and

2. The sequence must not have homology with or be predicted to hybridize with any known/published gene or expressed sequence tag (EST).

PCR primer pairs are designed for the selected sequence(s) and PCR is performed using genomic DNA (as a template) to generate PCR fragments (double strand DNA) corresponding to the non-transcribed sequence(s) as the control DNA. Additional control DNA can be cloned using a vector and standard techniques. Subsequently, standard techniques such as in vitro transcription are used to generate mRNA (complementary to the cDNA and containing a poly-A tail) as the control mRNA. Standard techniques are used for purifying the Control DNA and Control mRNA products, and for estimating their concentrations.

Empirical testing is also performed to ensure lack of hybridization between the Control DNA on the array and other mRNAs, as well as with mRNA from important gene expression systems (e.g., human, mouse, Arabidopsis, etc.).

The above approaches were used to generate twenty-three universal control sequences from intergenic regions of the yeast Saccharomyces cerevisiae genome. Specifically, using yeast genome sequence data publicly available (http://genome-www.stanford.edu/Saccharomyces/), intergenic regions approximately 1 kb in size were identified. These sequences were BLAST'd and those showing no homology to other sequences were identified as candidates for artificial gene controls. Candidates were analyzed for GC-content and a subset with a GC-content of ≧36% was identified. Specific primer sequences have been identified and primers synthesized. PCR products amplified with the specific primers have been cloned directly into the PGEM™-T Easy vector (Promega Corp., Madison, Wis.). Both array targets and templates for spike mRNA have been amplified from these clones using distinct and specific primers.

A greater number of intergenic regions have been cloned for testing. DNA samples from all the candidates were amplified, spotted on glass microarray slides and hybridized with mRNA samples from several species and each candidate spike mRNA, respectively, to identify those that do not cross-hybridize. First, they were screened for no cross-hybridization with RNA from different biological species. mRNA from human (eight tissues: skeletal muscle, spleen, liver, heart, kidney, brain, placenta and lung), mouse (six tissues: skeletal muscle, spleen, liver, heart, kidney and brain), rat (six tissues: skeletal muscle, spleen, liver, heart, kidney and brain), yeast (S. cerevisiae) and bacteria (E. coli and two Archaea species), as well as total RNA from plant (Arabidopsis, Oil Palm) were tested against the control candidates. Candidates that did not cross-react with the RNA samples from the species tested were then selected for cross-hybridization with each other. The candidates were hybridized with each candidate mRNA independently.

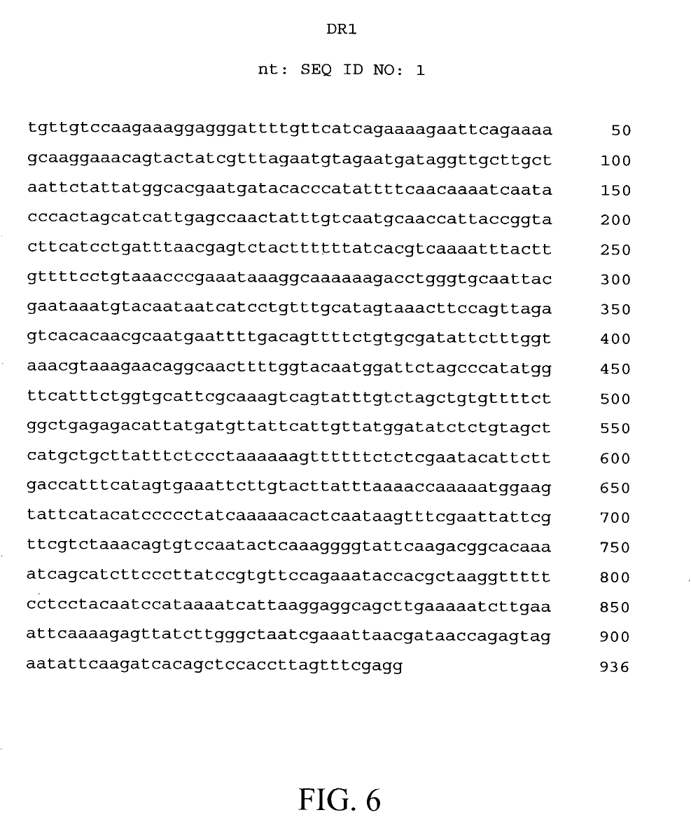

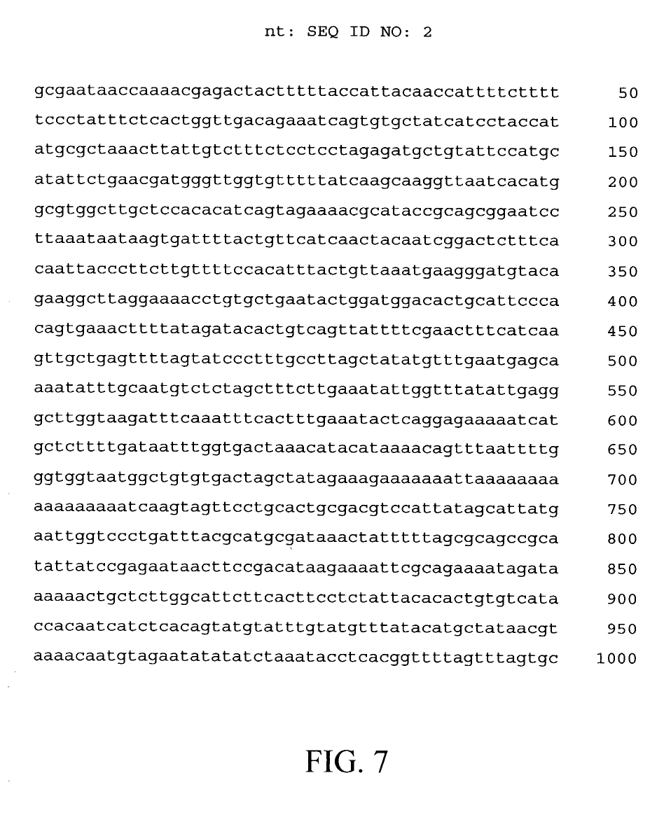

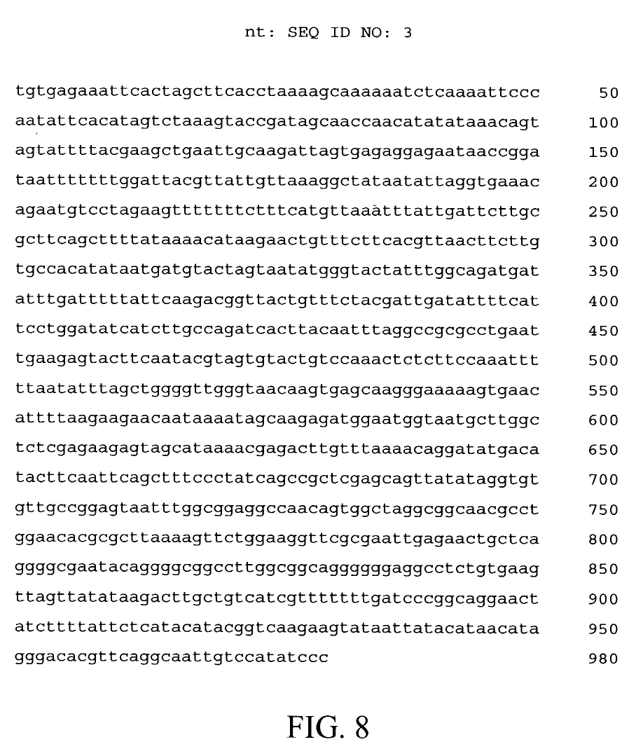

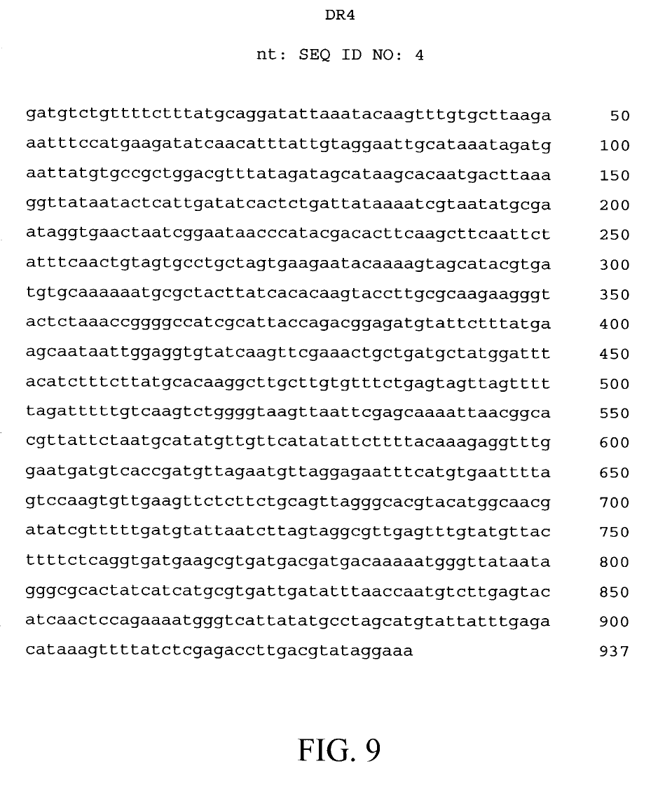

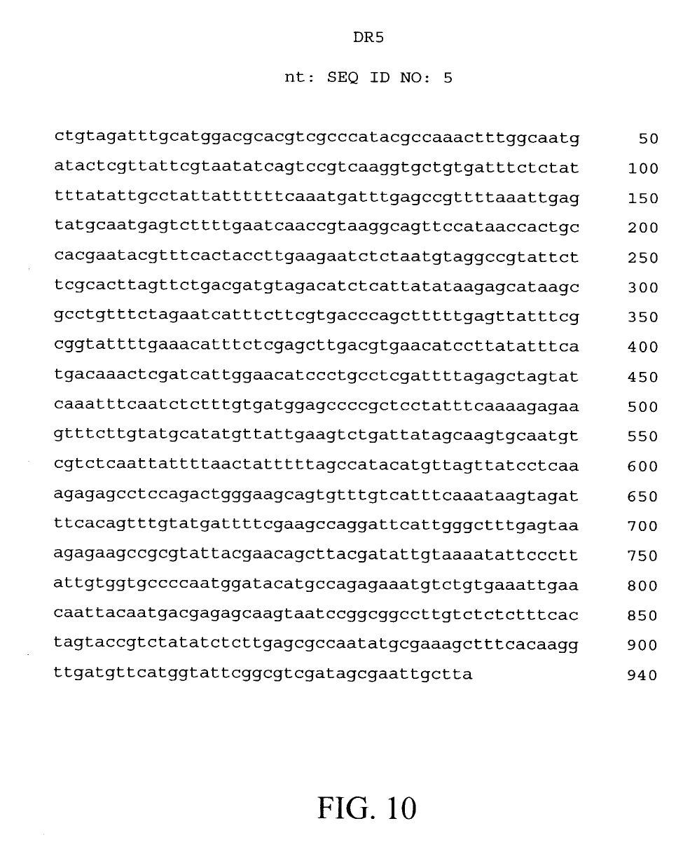

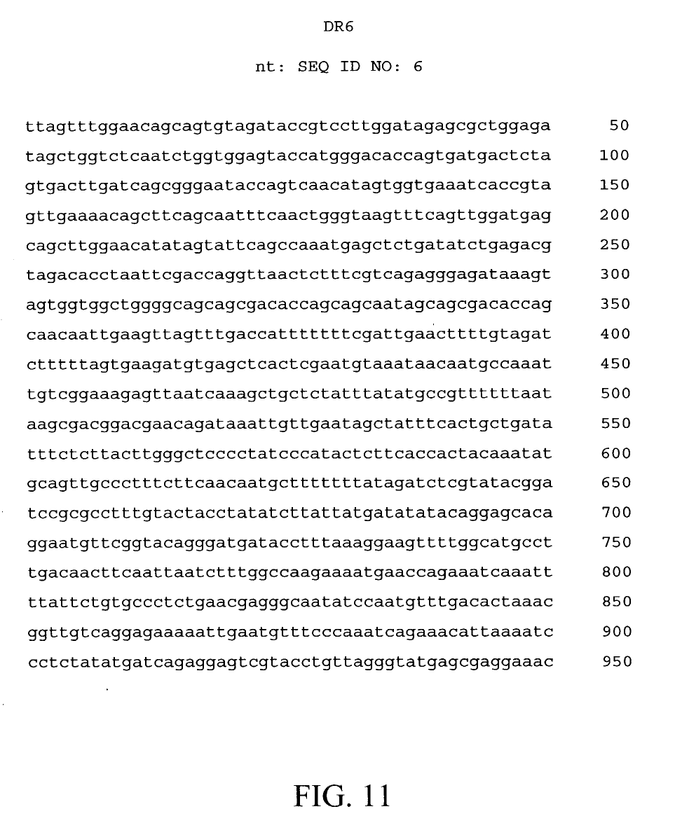

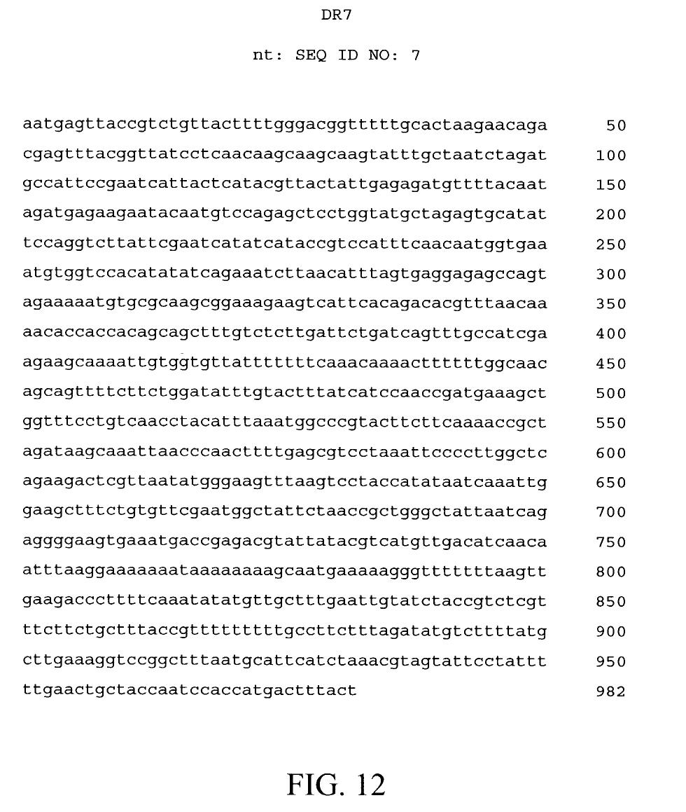

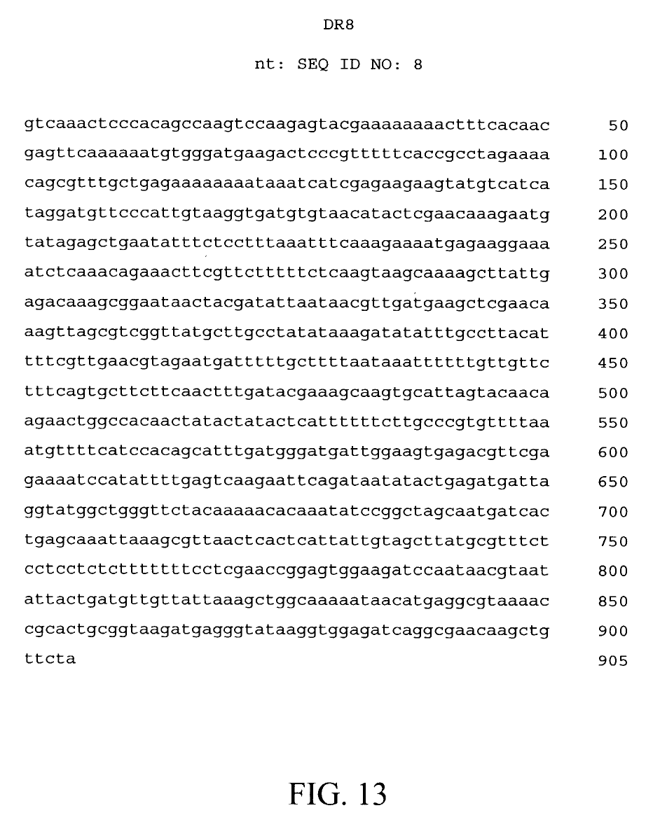

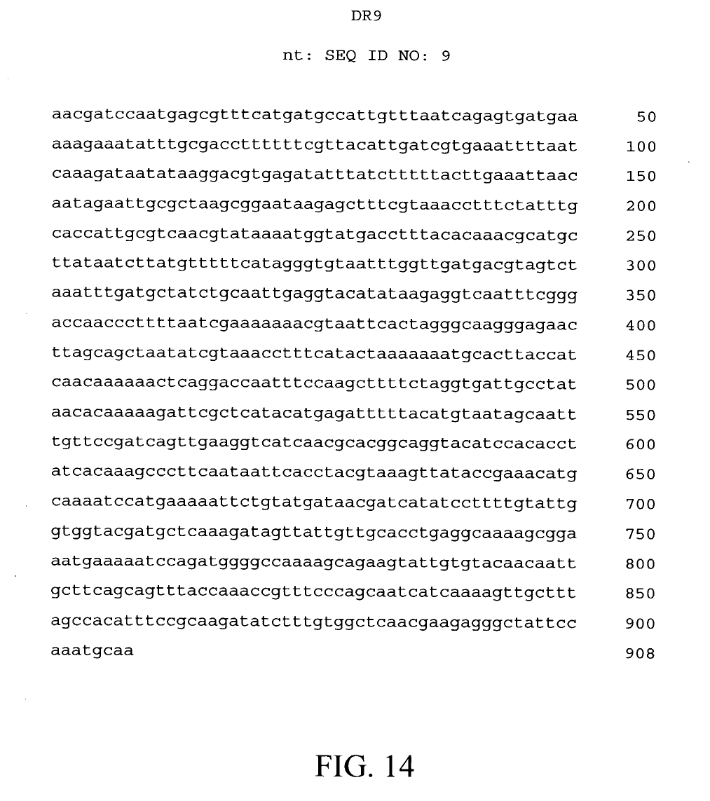

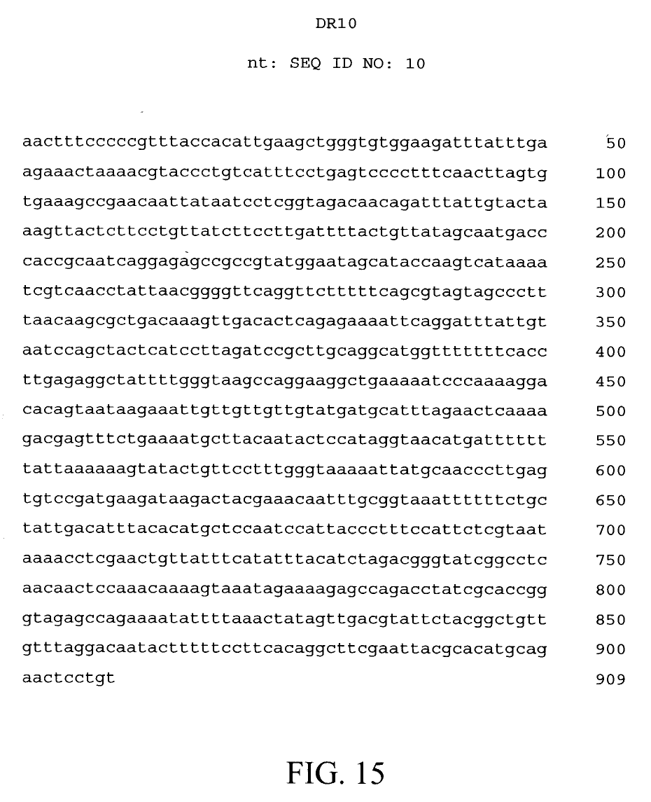

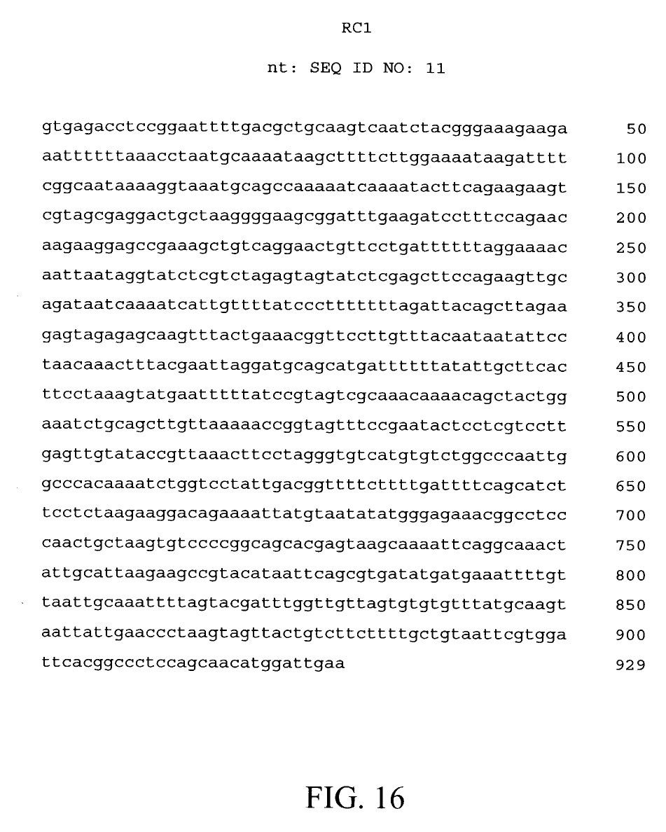

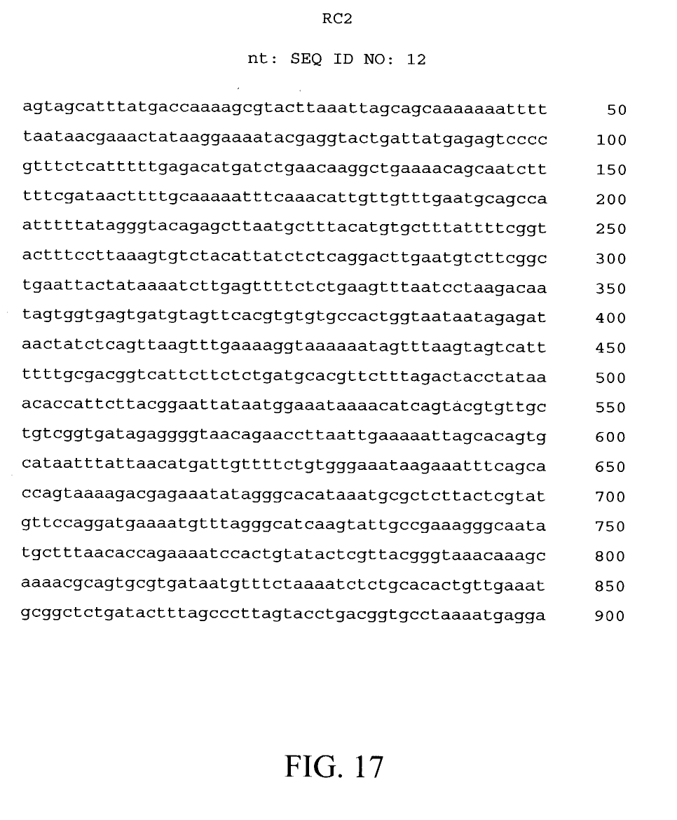

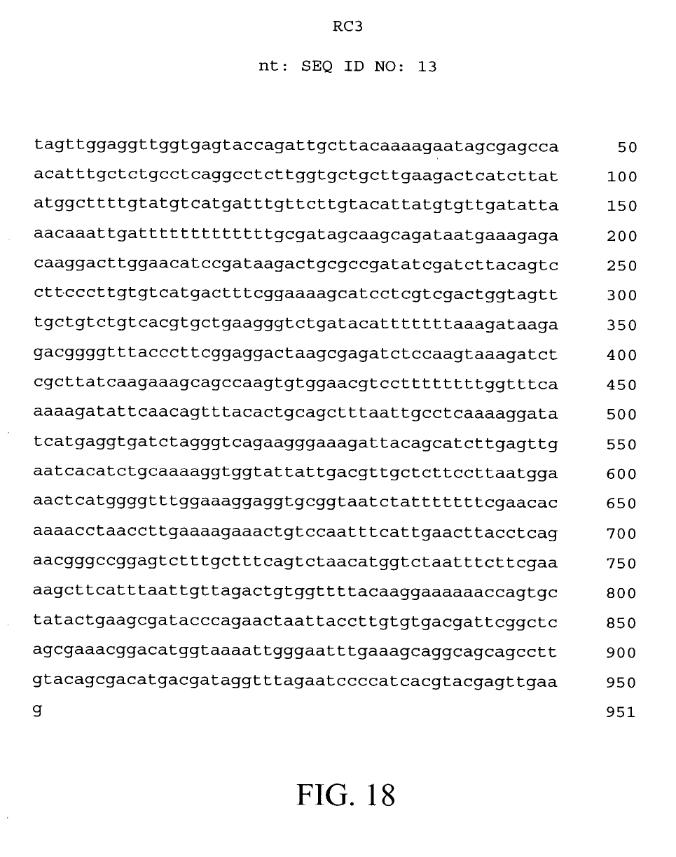

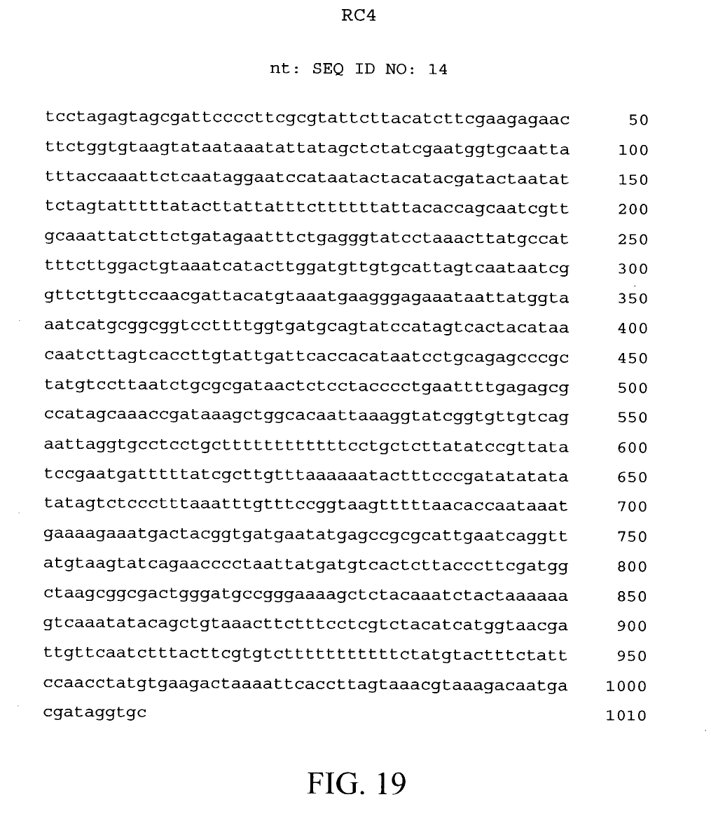

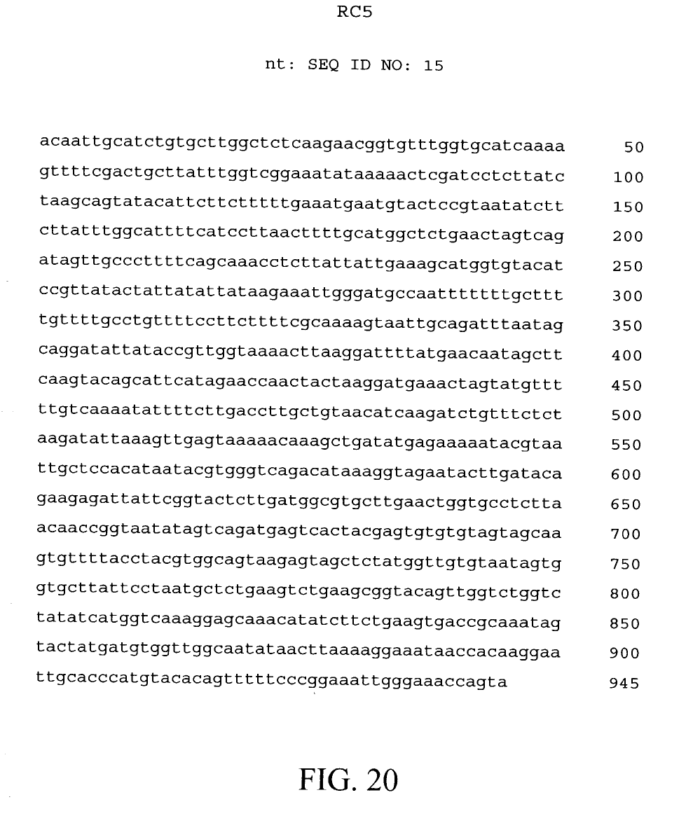

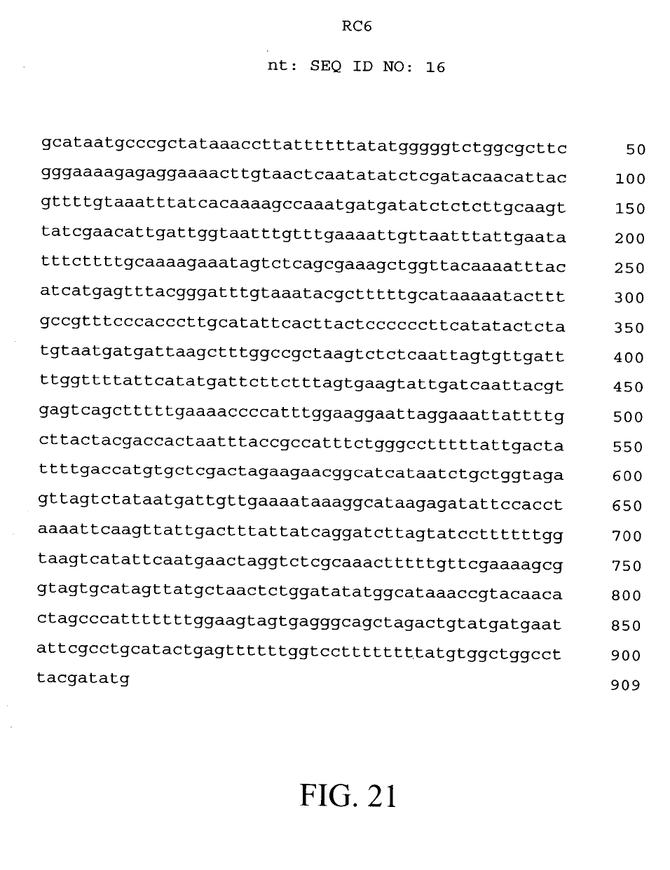

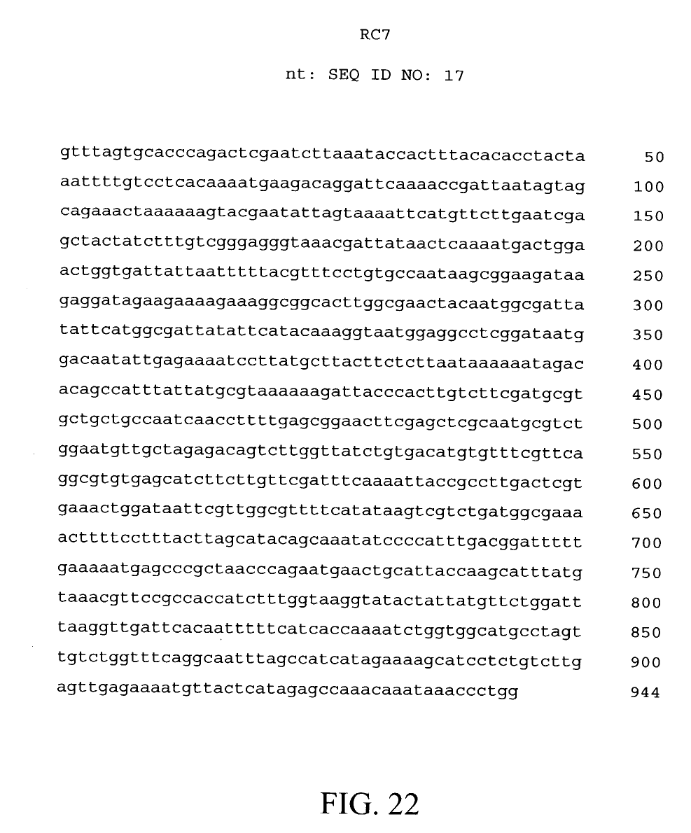

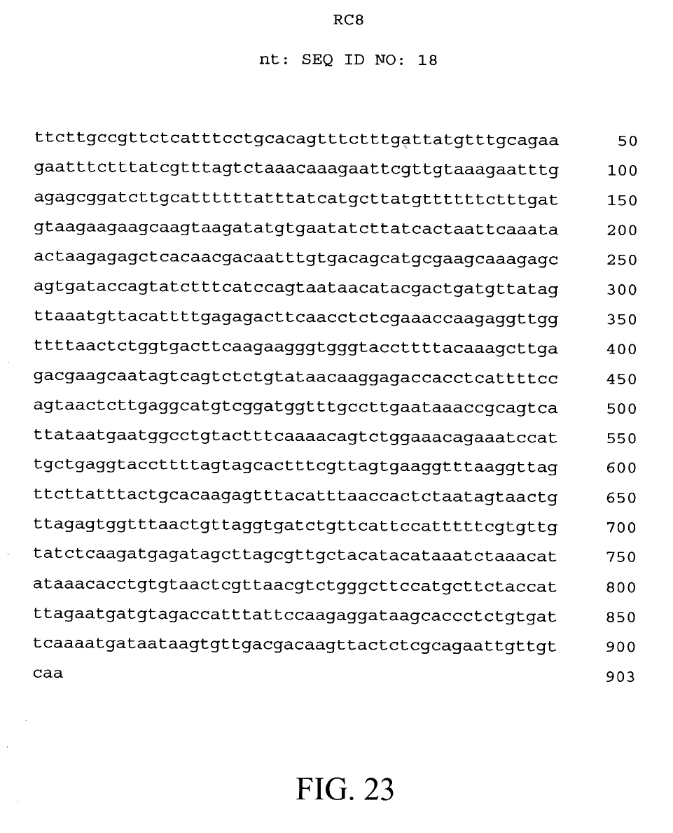

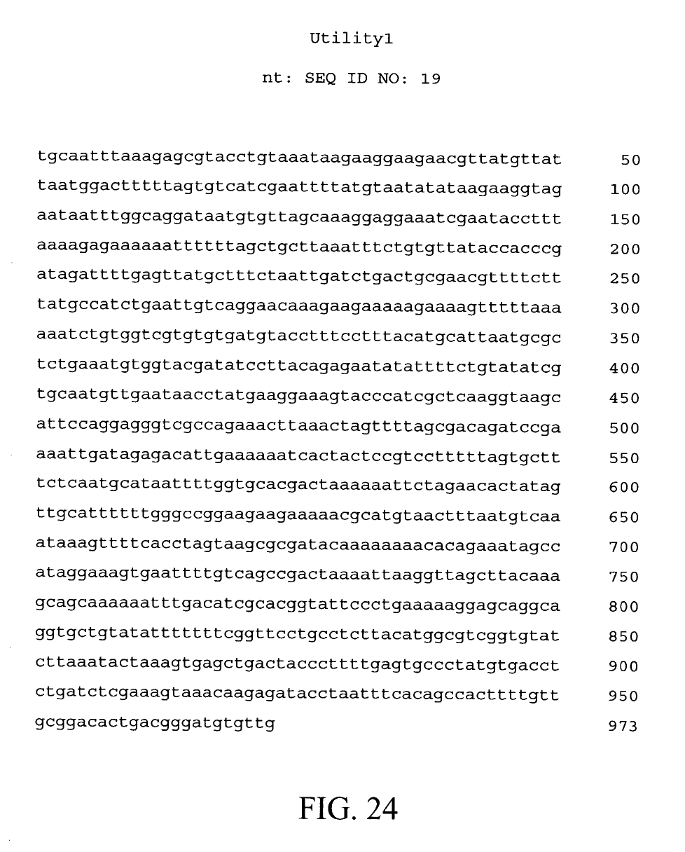

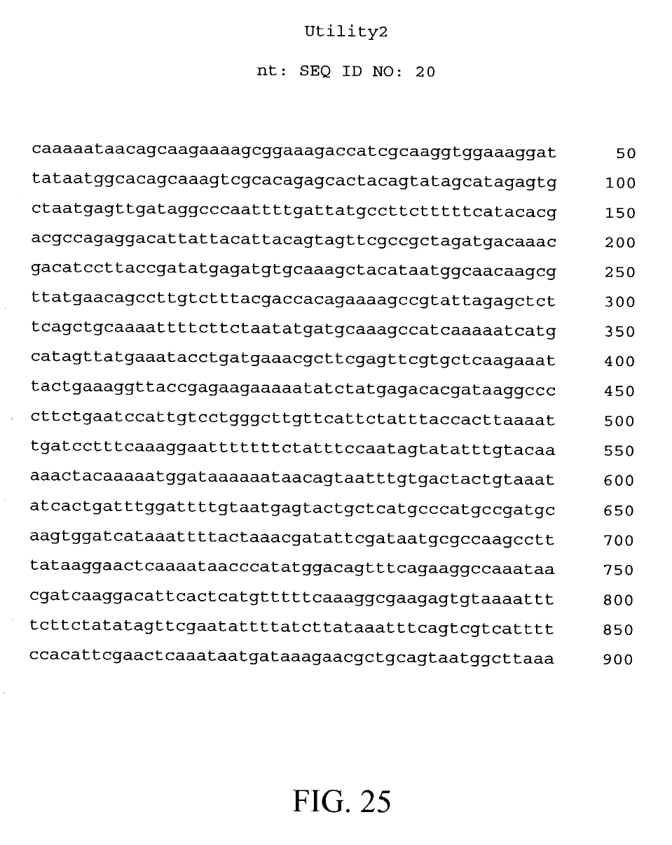

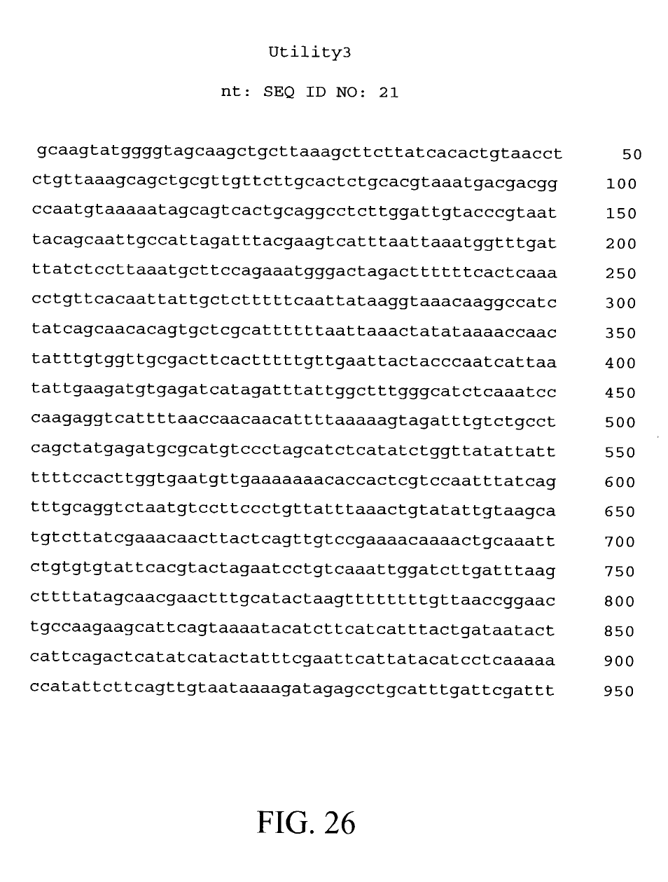

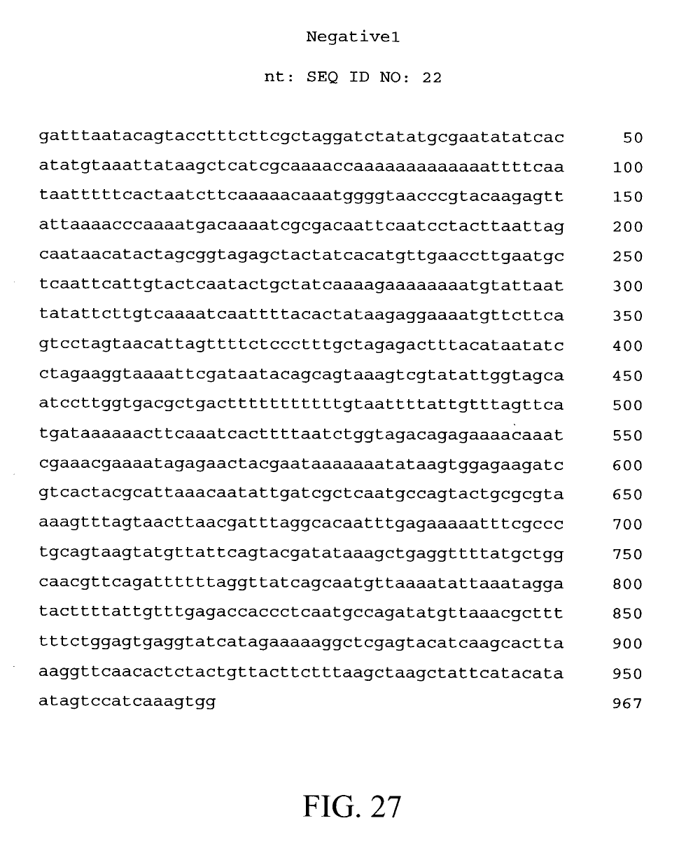

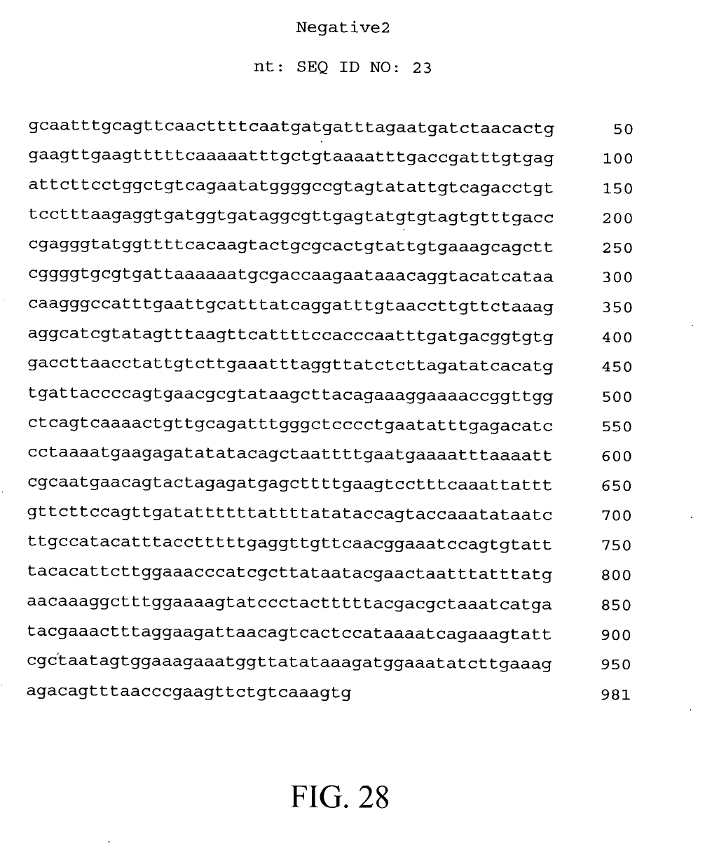

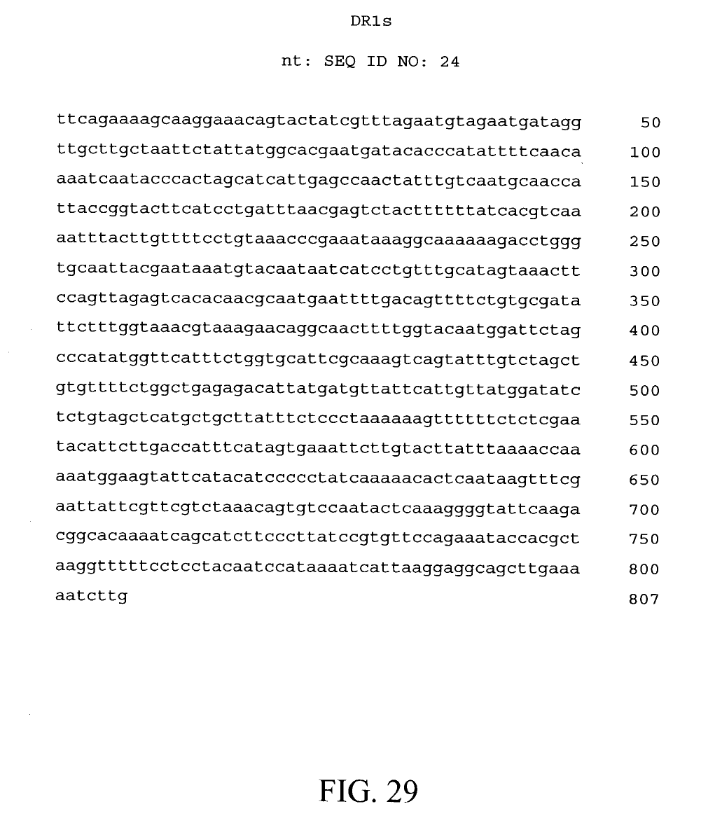

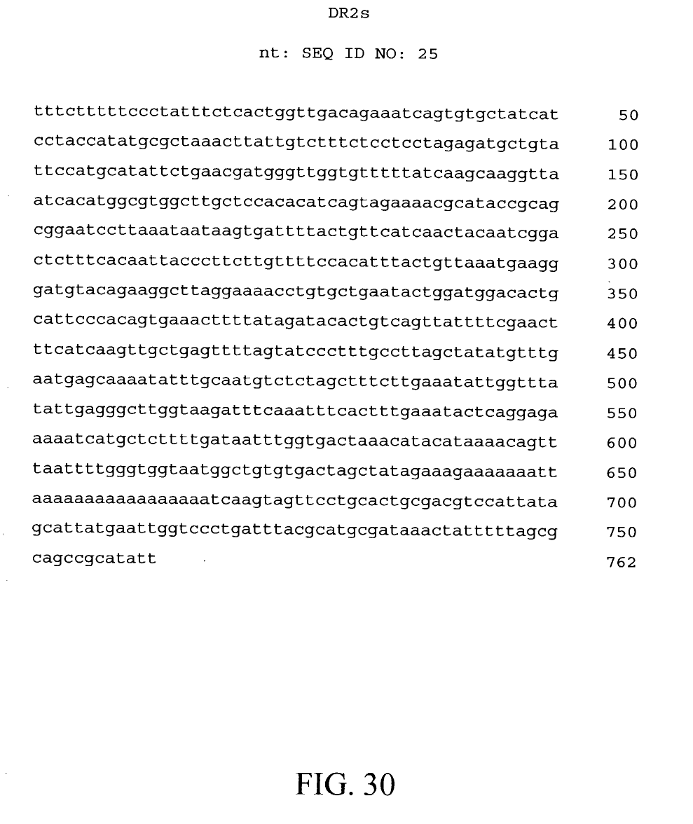

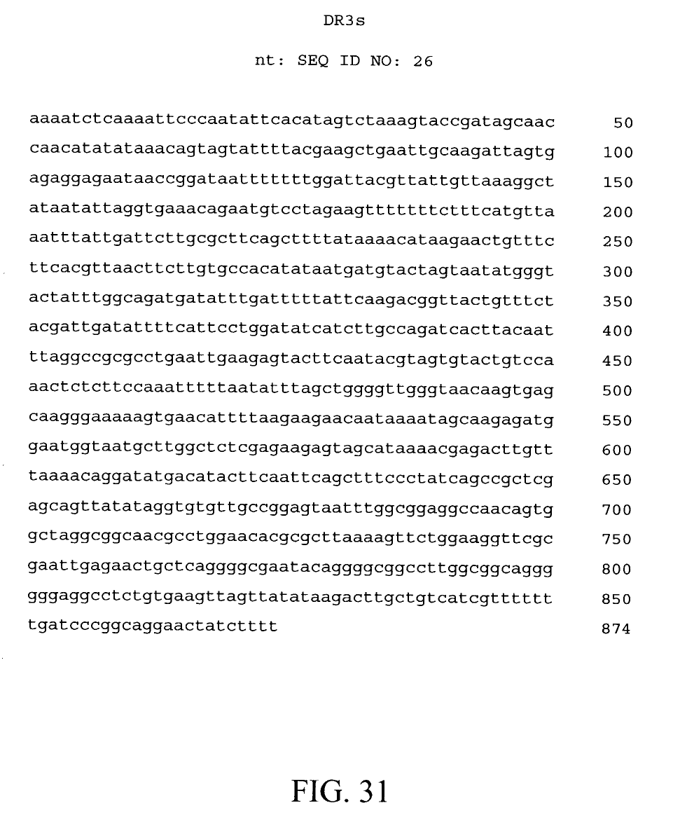

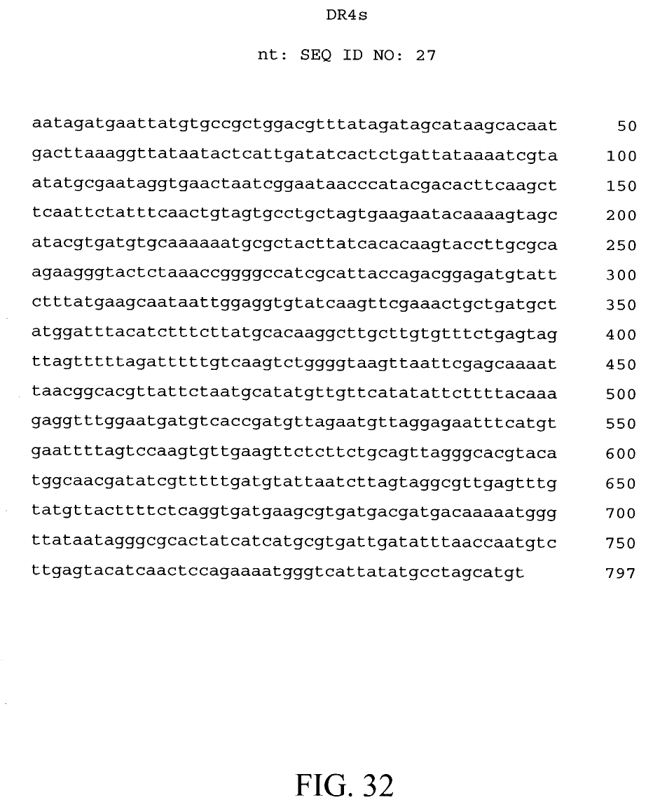

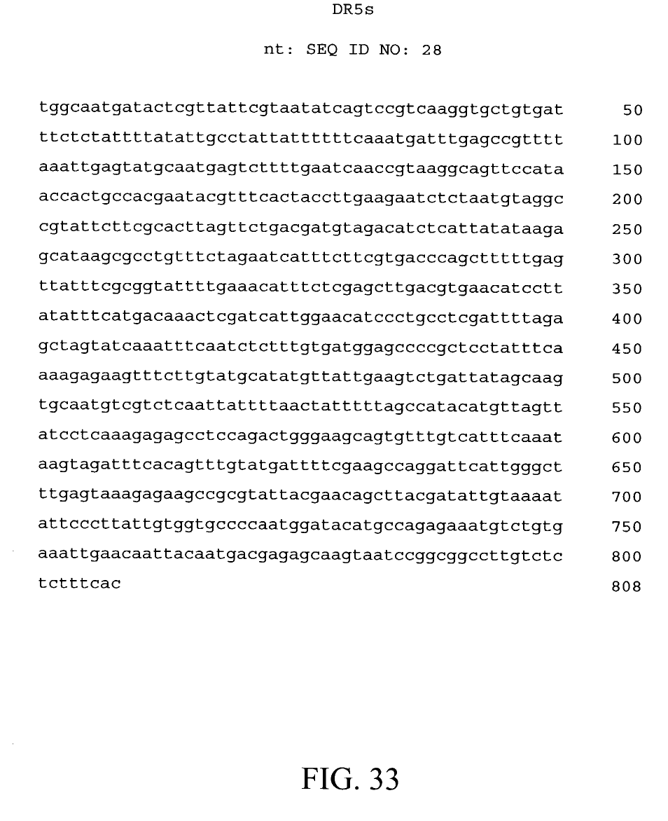

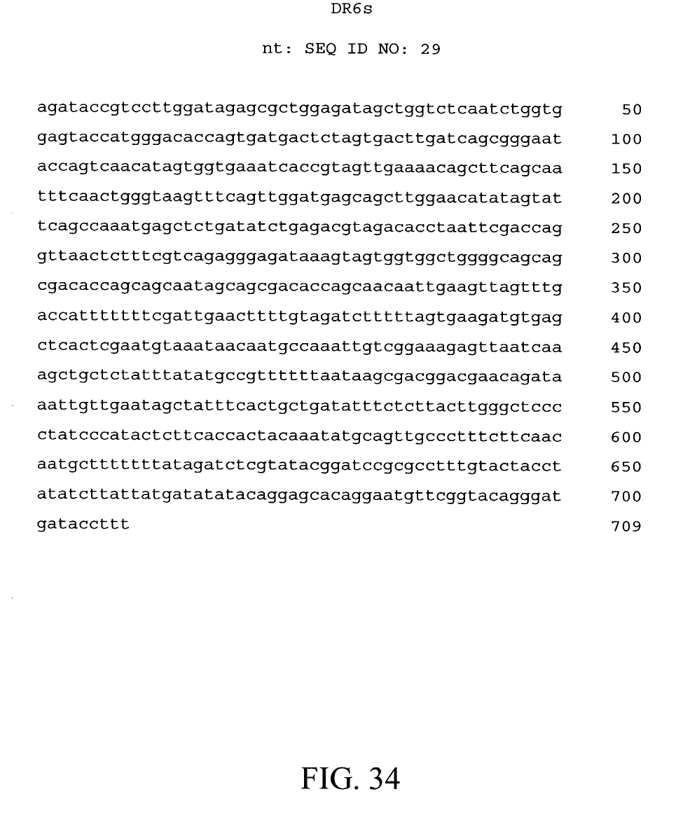

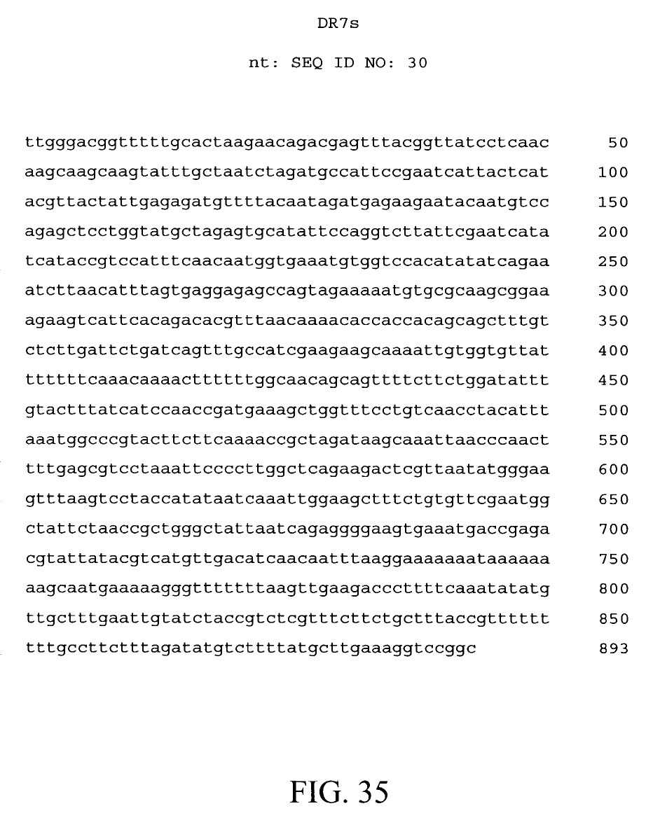

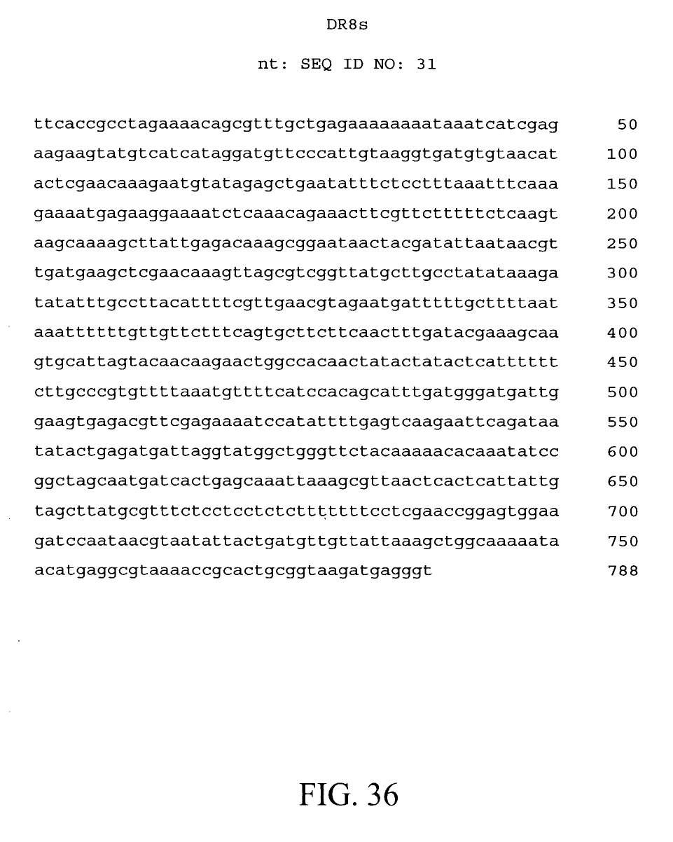

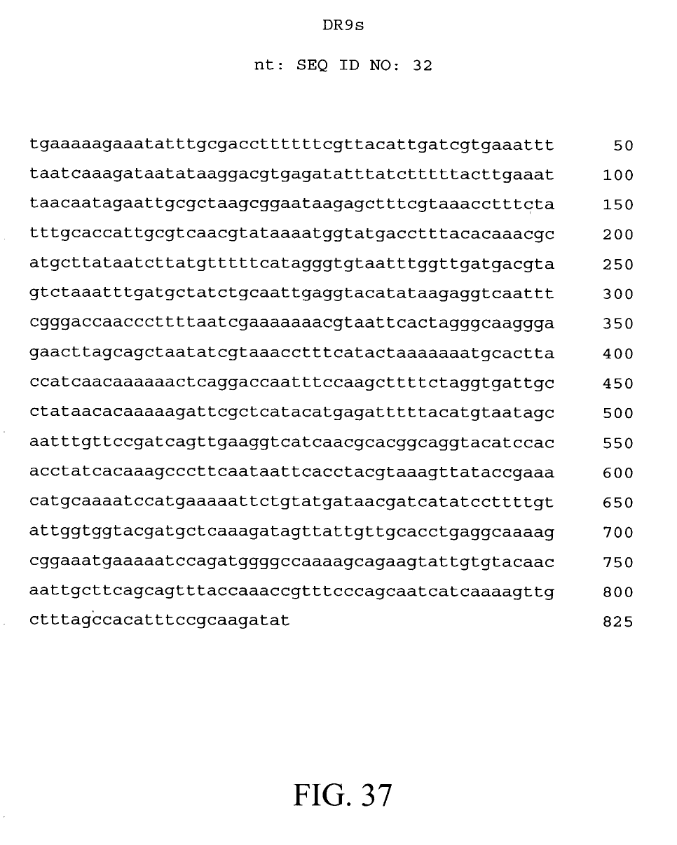

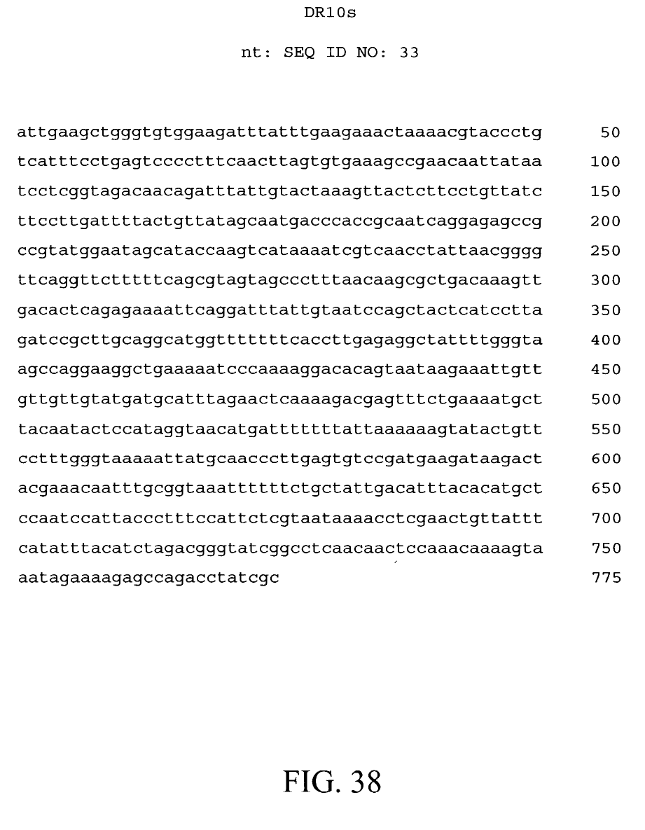

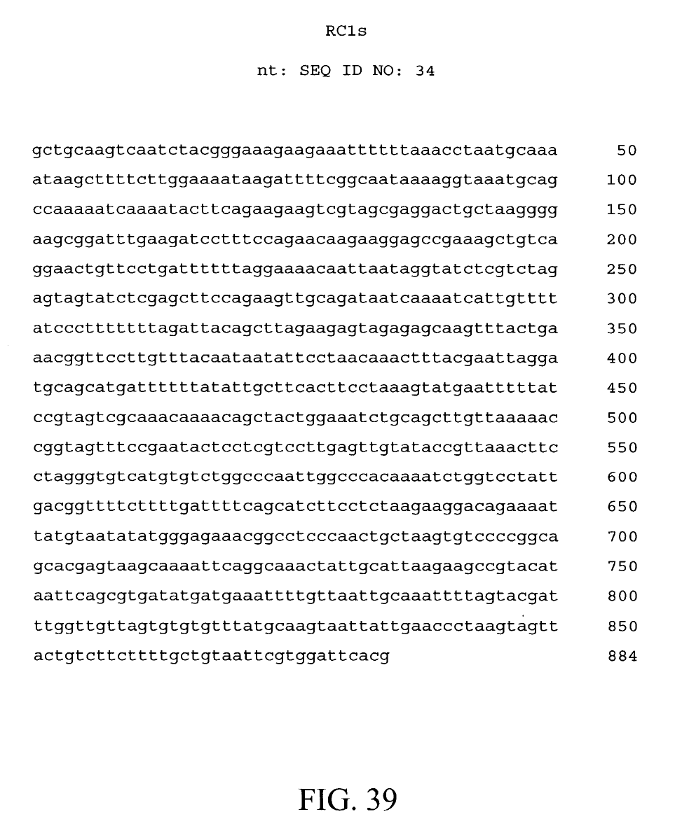

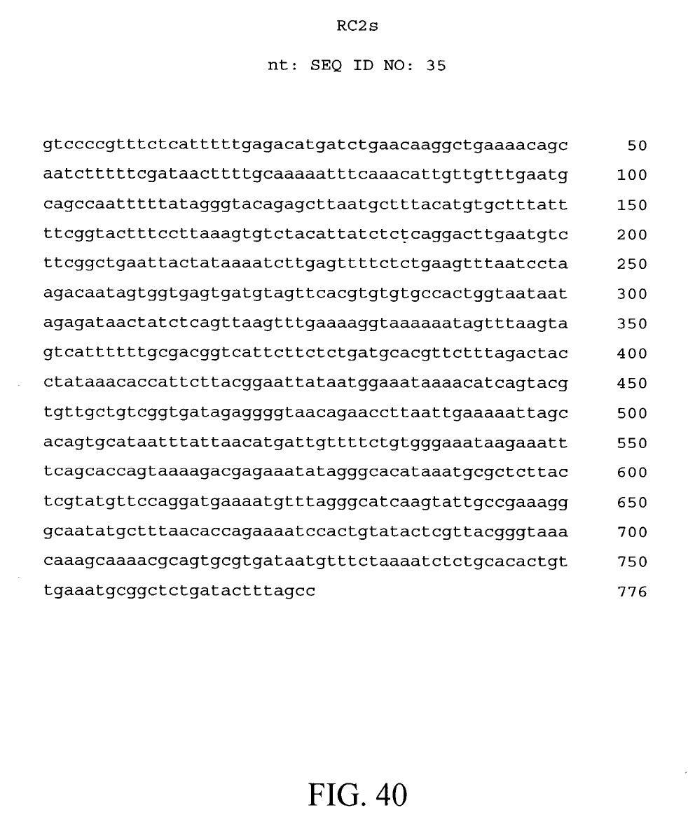

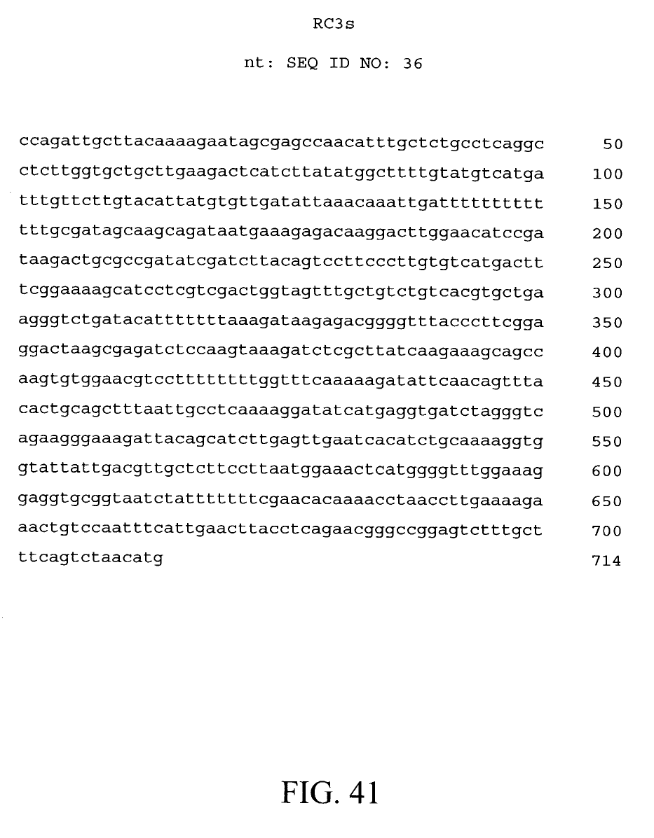

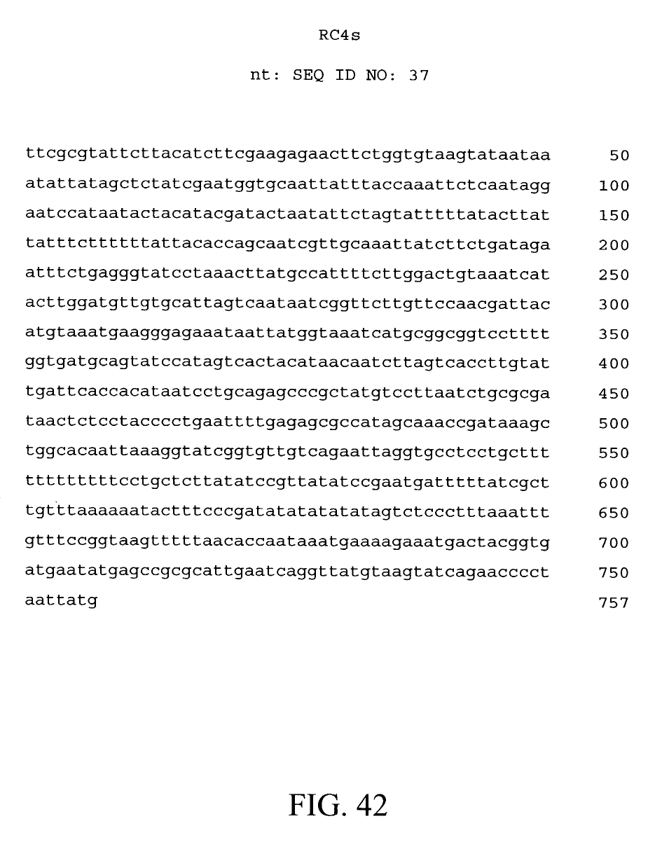

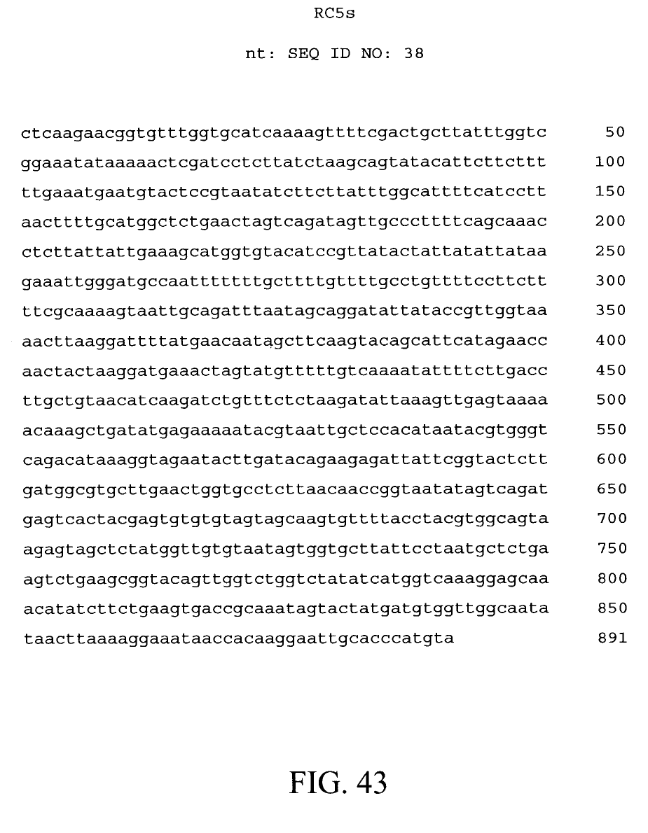

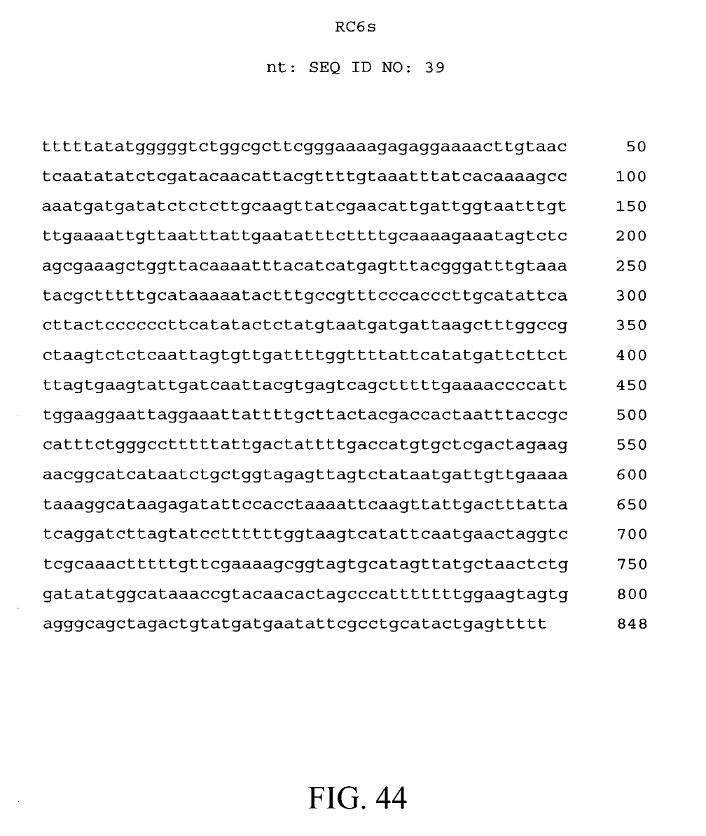

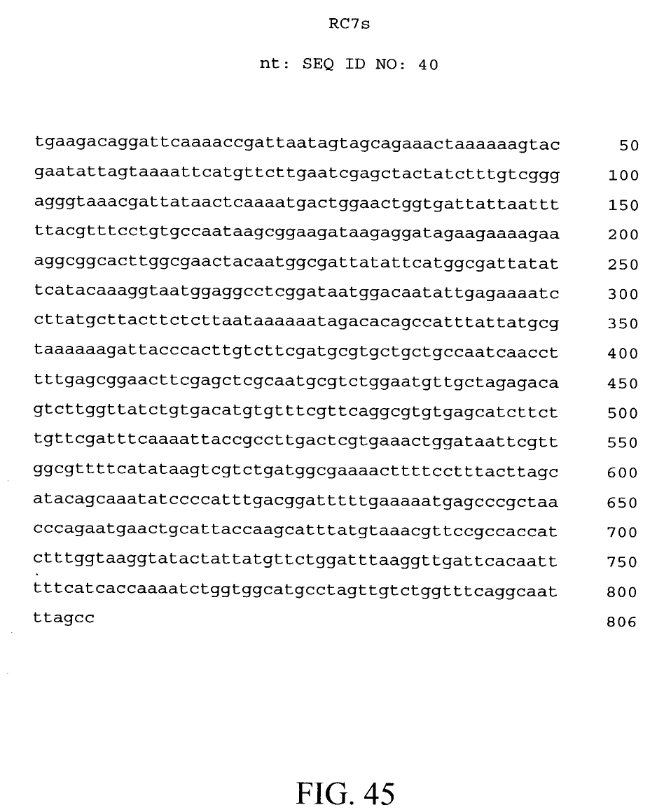

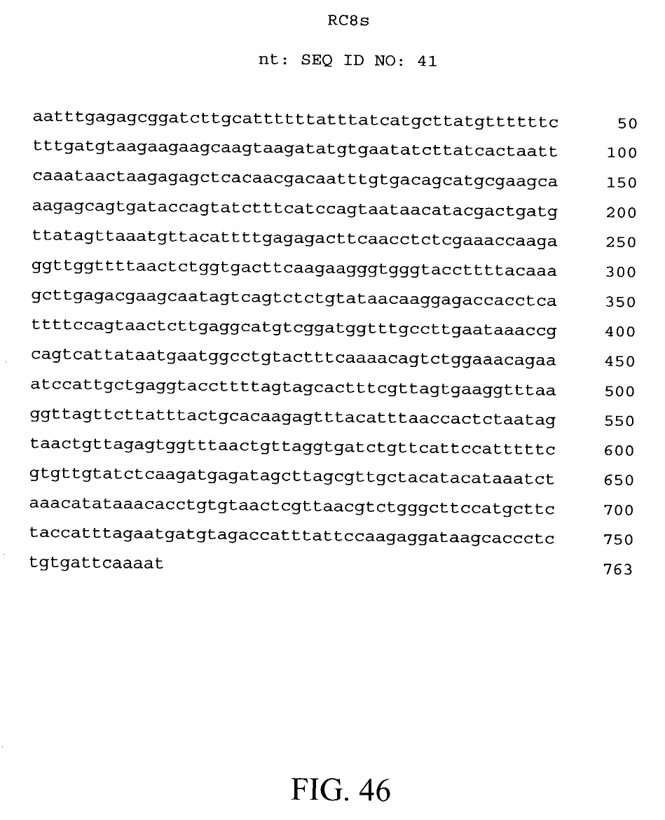

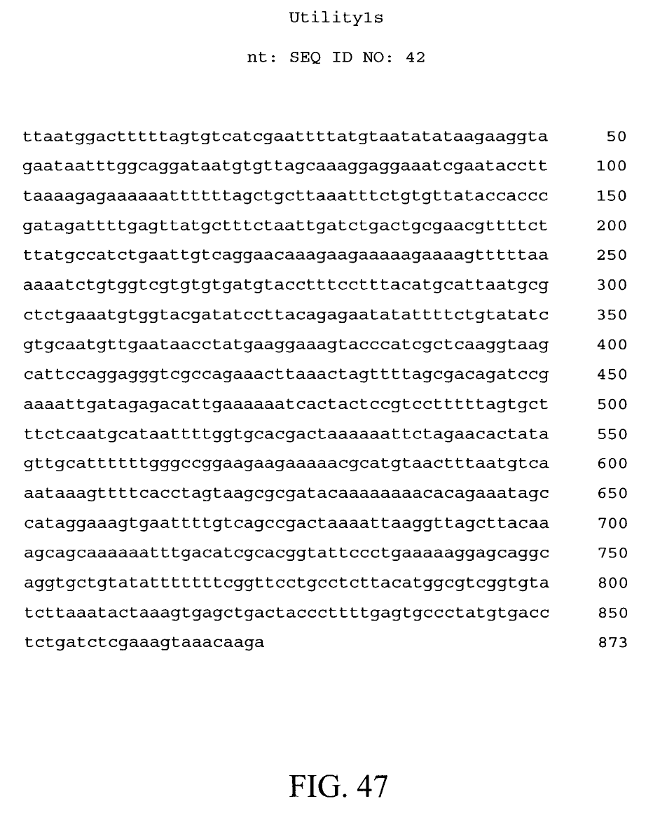

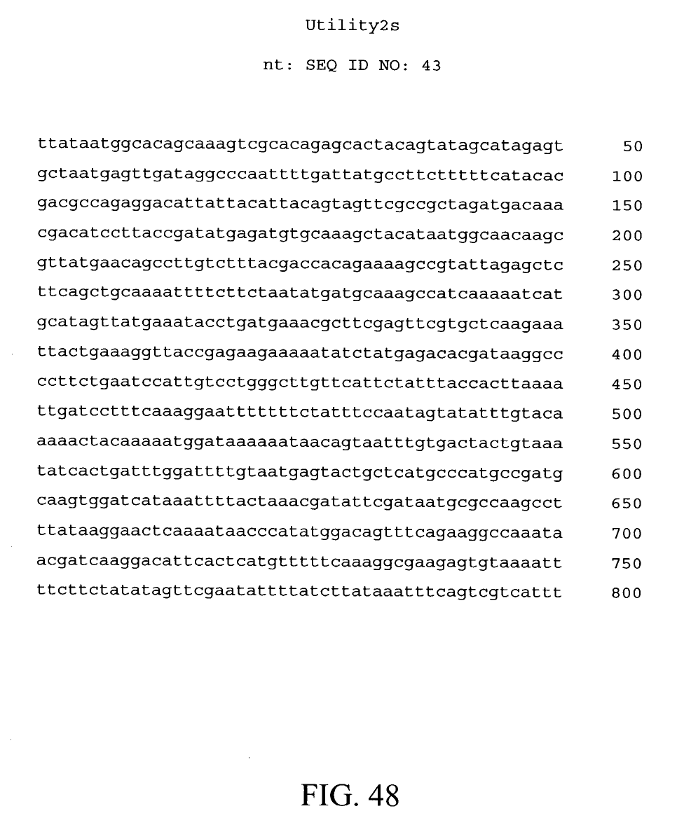

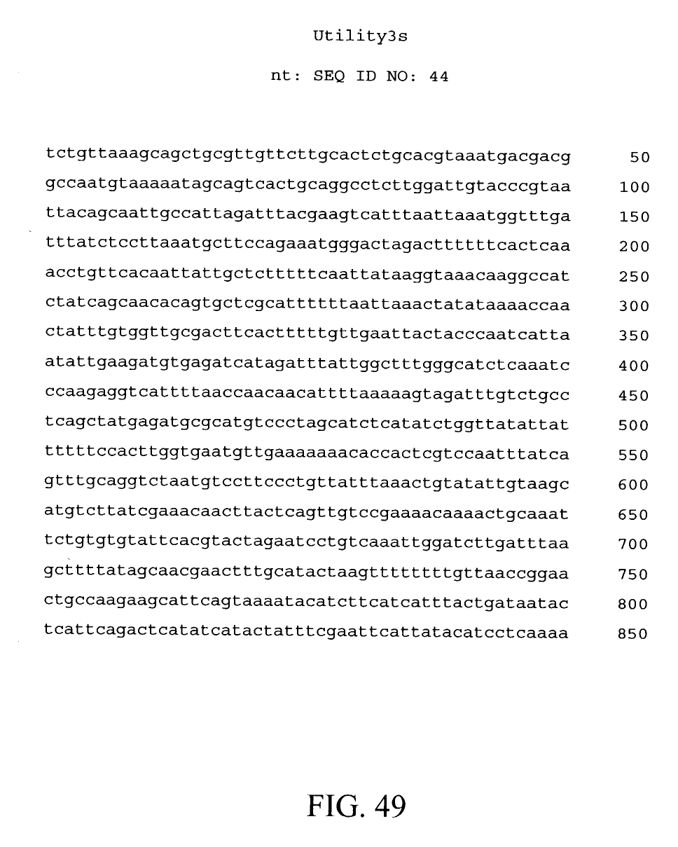

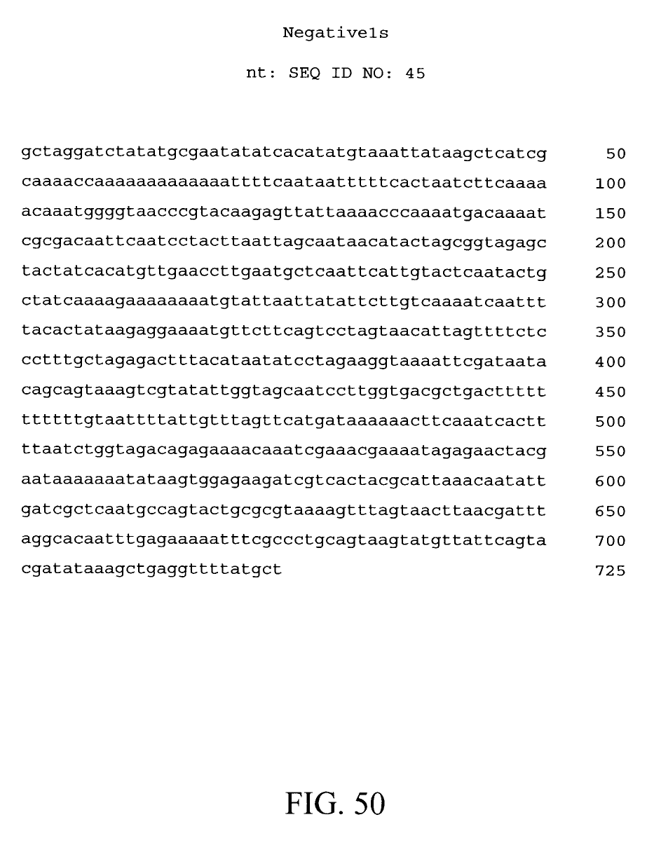

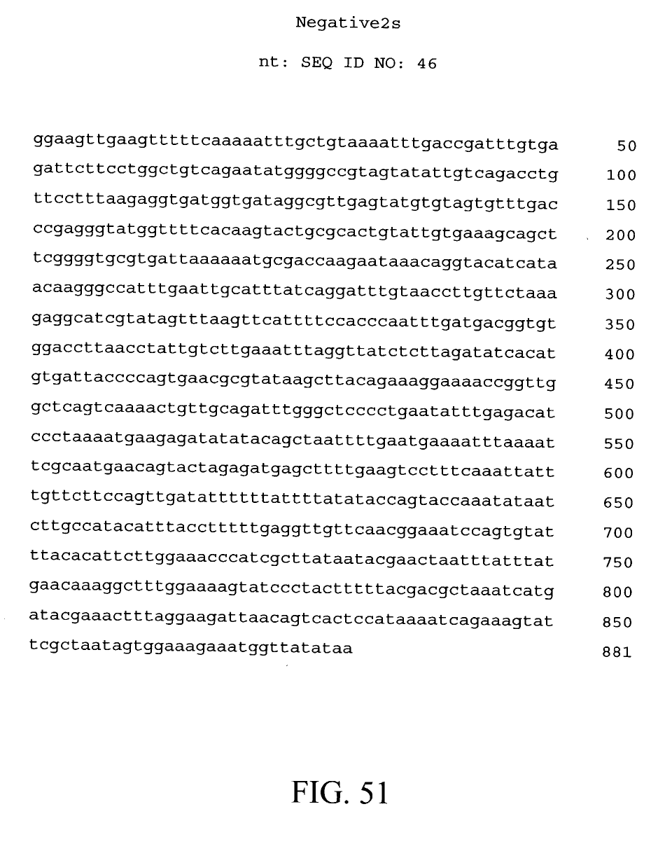

From the candidate clones that exhibited specific hybridization, twenty-three were included into the final set of universal controls. FIG. 6 through FIG. 28 presents the nucleotide sequences of the twenty-three controls spotted on the microarray slides, while FIGS. 29 through 51 presents the nucleotide sequences of the twenty-three controls that were transcribed and used in a spike mix, respectively. SEQ ID NO: 1 through SEQ ID NO: 23 present the nucleotide sequences of the twenty-three controls spotted on the microarray slides, while SEQ ID NO: 24 through SEQ ID NO: 46 present the nucleotide sequences of the controls that were transcribed and used in a spike mix.

These universal controls, when included in microarray experiments, perform as:

1. Negative controls: Control DNA included in the array, but for which no complementary artificial mRNA is spiked into the RNA sample, serves as a negative control;

2. Calibration controls: Several different Control DNA samples may be included in an array, and the complementary Control mRNA for each is included at a known concentration, each having a different concentration of mRNA. The signals from the array features corresponding to these Controls or Calibrators may be used to construct a “dose-response curve” or calibration curve to estimate the relationship between signal and amount of mRNA from the sample;

3. Ratio controls: In two-color microarray gene expression studies, it is possible to include different, known, levels of Control mRNA complementary to Control DNA in the labeling reaction for each channel. The ratio of signals for the two dyes from a particular gene can be compared to the ratio of signals from the two dyes of the Control mRNA. This can serve as a test of the accuracy of the system for determining gene expression ratios.

4.Utility controls: These controls can be added into the sample preparation steps (such as RNA extraction and purification) for normalization of the biological samples and assessment of sample losses during preparation. Alternatively, they can be added to labeling reactions as additional calibrators or ratios.

Mixtures of several different Control mRNA species can be prepared (spike mixes) at known concentrations and ratios to simplify and standardize the experimental protocol while providing a comprehensive set of precision and accuracy information. Table 1 demonstrates one embodiment of this concept. The mRNA from the final set of clones have been pre-mixed at specific concentrations and ratios so they can serve as the various controls when hybridized to their corresponding control DNA spotted on the arrays. Ten calibrators (those included in the labeling reaction at a ratio of 1:1) spanning a dynamic range of 4.5 orders of magnitude are included as calibration controls. Eight ratio controls are included, at two expression levels (low and medium to high) and reversed with respect to the reference and test samples.

The universal controls as shown in Table 1 can be used as references for microarray validation and standardization across biological species and experimental platforms. These controls can be used to verify the accuracy and precision of gene expression ratios, and the sensitivity and dynamic range of the microarray system. Through the use of Calibration (standard) curves, these controls may allow reporting gene expression levels in consistent mass units, improving the comparisons of results across laboratories.

The following examples demonstrate how these Control DNA and Control mRNA were generated, and then used as universal controls in microarray gene expression experiments. They are representative of the many different types of experiments that could benefit from the use of these controls. The following examples are offered by way of illustration and not by way of limitation.

TABLE 1
Suggested Control mRNA spike mix composition for
two-color gene expression ratio experiments.

| | | | |
|---|---|---|---|
| | | Target | mRNA in the Spike Mix |
| Control | Control | Cy3:Cy5 | (pg/2 μl of spike) |

| Type | Name | Ratio | Cy3 | Cy5 |
|---|---|---|---|---|
| Calibration | DR1s | 1:1 | 30 000 | 30 000 |
| Calibration | DR2s | 1:1 | 10 000 | 10 000 |
| Calibration | DR3s | 1:1 | 3 000 | 3 000 |
| Calibration | DR4s | 1:1 | 1 000 | 1 000 |
| Calibration | DR5s | 1:1 | 300 | 300 |
| Calibration | DR6s | 1:1 | 100 | 100 |
| Calibration | DR7s | 1:1 | 30 | 30 |
| Calibration | DR8s | 1:1 | 10 | 10 |
| Calibration | DR9s | 1:1 | 3 | 3 |
| Calibration | DR10s | 1:1 | 1 | 1 |
| Ratio | RC1s | 3:1 low | 300 | 100 |
| Ratio | RC2s | 1:3 low | 100 | 300 |
| Ratio | RC3s | 3:1 high | 3 000 | 1 000 |
| Ratio | RC4s | 1:3 high | 1 000 | 3 000 |
| Ratio | RC5s | 10:1 low | 300 | 30 |
| Ratio | RC6s | 1:10 low | 30 | 300 |
| Ratio | RC7s | 10:1 high | 10 000 | 1 000 |
| Ratio | RC8s | 1:10 high | 1 000 | 10 000 |
| Utility | Utility1s | User | User | User |
| | | defined | defined | defined |
| Utility | Utility2s | User | User | User |
| | | defined | defined | defined |
| Utility | Utility3s | User | User | User |
| | | defined | defined | defined |
| Negative | Negative1s | NA | 0 | 0 |
| Negative | Negative2s | NA | 0 | 0 |

### EXAMPLE 1

### Generation of Artificial Controls from Intergenic Regions of S. cerevisiae Genome

Using yeast genomic sequence data publicly available (http://genome-www.stanford.edu/Saccharomyces/), intergenic regions (YIRs) approximately 1 kb in size were identified. These sequences were BLAST'd and those showing no homology to other sequences were identified as candidates for artificial gene controls. Candidates were analyzed for GC-content and a subset with a GC-content of ≧36% was identified. Specific primer sequences have been identified and synthesized. PCR products amplified with the specific primers have been cloned directly into the pGEM™-T Easy vector (Promega Corp., Madison, Wis.). Both array targets and templates for spike mRNA have been amplified from these clones using distinct and specific primers.

When used as DNA controls, the YIR sequences were amplified by PCR with specific primers, using 5 ng of cloned template (plasmid DNA) and a primer concentration of 0.5 μM in a 100 μl reaction volume, and cycled as follows: 35 cycles of 94° C. 20 sec., 52° C. 20 sec., 72° C. 2 min., followed by extension at 72° C. for 5 min.

All YIR control mRNAs for the spike mix are generated by in vitro transcription. Templates for in vitro transcription (IVT) are generated by amplification with specific primers that are designed to introduce a T7 RNA polymerase promoter on the 5′ end and a polyT (T21) tail on the 3′ end of the PCR products. Run-off mRNA is produced using 1 μl of these PCR products per reaction with the AmpliScribe system (Epicentre, Madison, Wis.). IVT products are purified using the RNAEasy system (Qiagen Inc., Valencia, Calif.) and quantified by spectrophotometry.

Initially, fifty intergenic region sequences have been cloned for testing. DNA samples from all the candidates were amplified, spotted on glass microarray slides and hybridized with mRNA samples from several species and each candidate spike mRNA, respectively, to identify those that do not cross-hybridize. First, they were screened for no cross-hybridization with RNA from different biological species. mRNA from human (8 tissues: skeletal muscle, spleen, liver, heart, kidney, brain, placenta and lung), mouse (6 tissues: skeletal muscle, spleen, liver, heart, kidney and brain), rat (6 tissues: skeletal muscle, spleen, liver, heart, kidney and brain), yeast (S. cerevisiae) and bacteria (E. coli and two Archaea species), as well as total RNA from plant (Arabidopsis, Oil Palm) were tested against the control candidates.

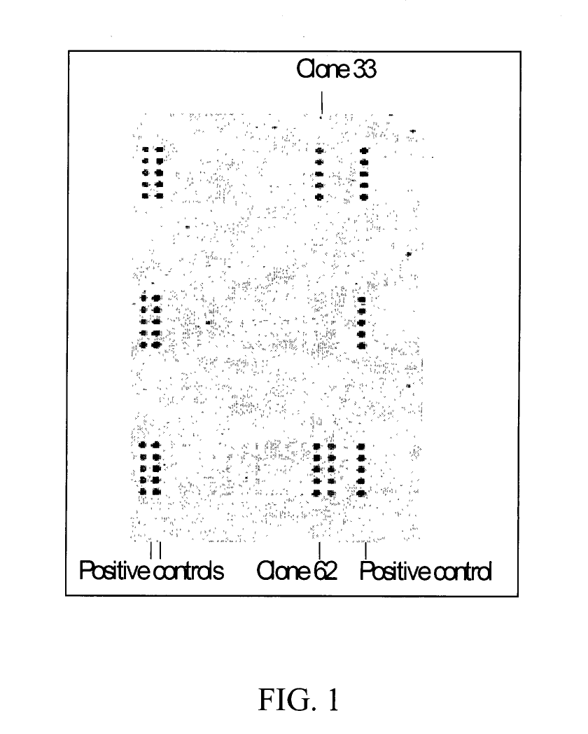

FIG. 1 shows the hybridization of candidates with human brain mRNA. The results indicated that two YIR clones, 33 and 62, hybridized with human brain RNA while the other candidates did not (since no appreciable signal is detected). Clones, such as 33 and 62, that exhibited such cross-hybridization were removed from the set of candidates for universal controls.

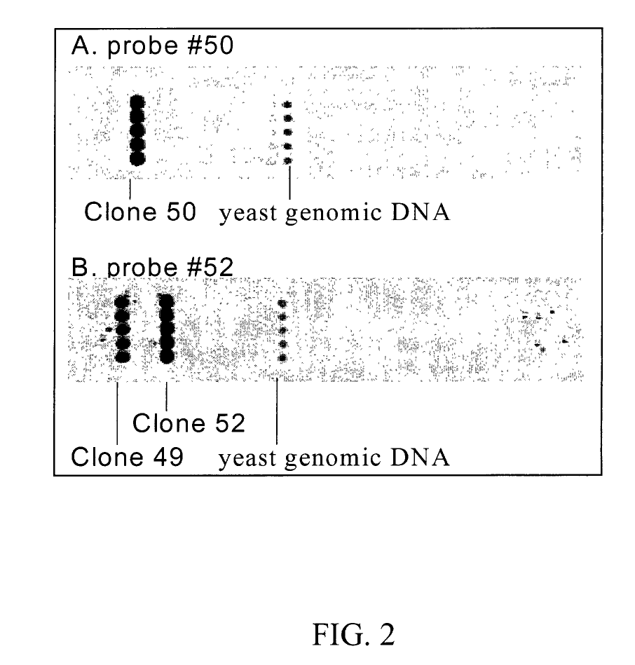

Candidates that did not cross-react with the RNA samples from the species tested were then tested for cross-hybridization with each other. The candidates were hybridized with each candidate mRNA independently. In FIG. 2 the labeled mRNA made from clone #50 was specifically hybridized against all other candidate clones. It hybridized only to its corresponding target DNA and can be included into the candidate set. However, clone #52 bound to the spot of clone #49 besides its own and therefore was not included in the candidate set.

From the candidate clones that exhibited specific hybridization, twenty-three are included into the final set of universal controls. FIG. 6 through FIG. 28 presents the nucleotide sequences of the twenty-three controls spotted on the microarray slides, while FIGS. 29 through 51 presents the nucleotide sequences of the twenty-three controls as used in a spike mix, respectively. The sequences of these clones are further presented in the Sequence Listing, incorporated herein by reference in its entirety, as follows:

SEQ ID NOs: 1-23 (nt, control nucleotide sequences, including calibration controls 1 through 10, ratio controls 1 through 8, utility controls 1 through 3, and negative controls 1 and 2 respectively);

SEQ ID NOs: 24-46 (nt, spike mix nucleotide sequences, including calibration controls 1 through 10, ratio controls 1 through 8, utility controls 1 through 3, and negative controls 1 and 2 respectively);

Upon confirmation of the exact structure, each of the above-described nucleic acids of confirmed structure is recognized to be immediately useful as a control.

### EXAMPLE 2

### Performance Evaluation of the Artificial Controls

The universal controls (both the spike mixes and their corresponding spotting samples) have been evaluated for their performance in real microarray experiments and tested for the following.

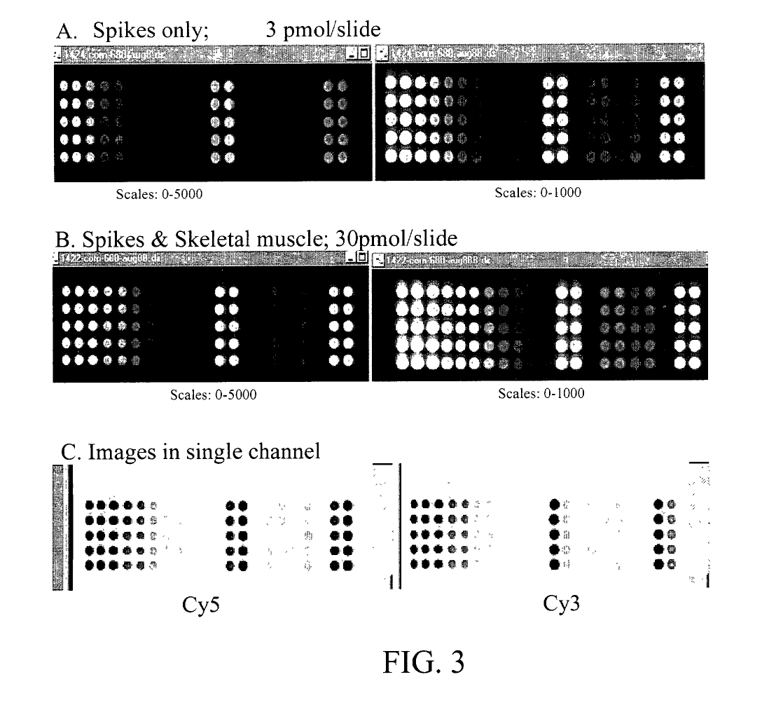

Experimental design, including array design and the hybridization sample concentration were tested (FIG. 3). Control samples were spotted in five replicates and hybridized with probes prepared with the spike mix only or the spike mixes with skeletal muscle mRNA. The same array image in FIG. 3 is shown at two different gray scales for easy visualization of signals across the entire dynamic range.

Universal utility, including hybridization of the spikes on pre-arrayed slides from various species were also tested. The controls showed no cross-hybridization on human, rat, mouse, Arabidopsis, Yeast and E. coli pre-arrayed slides from commercial sources (data not shown).

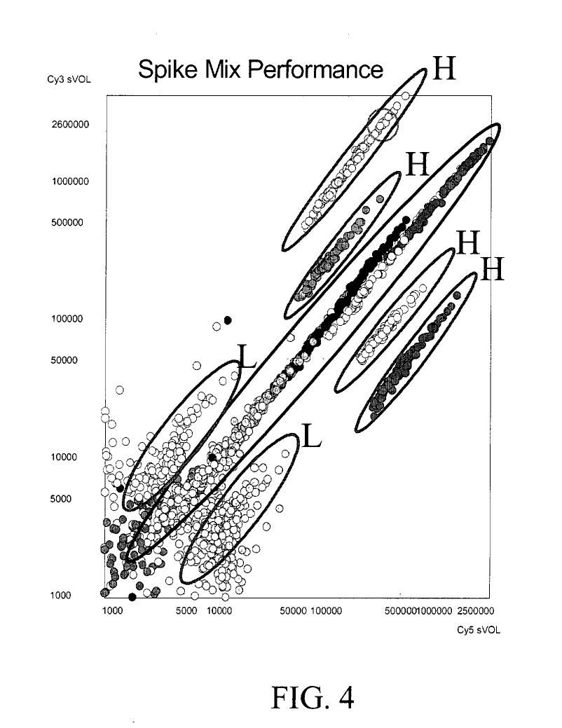

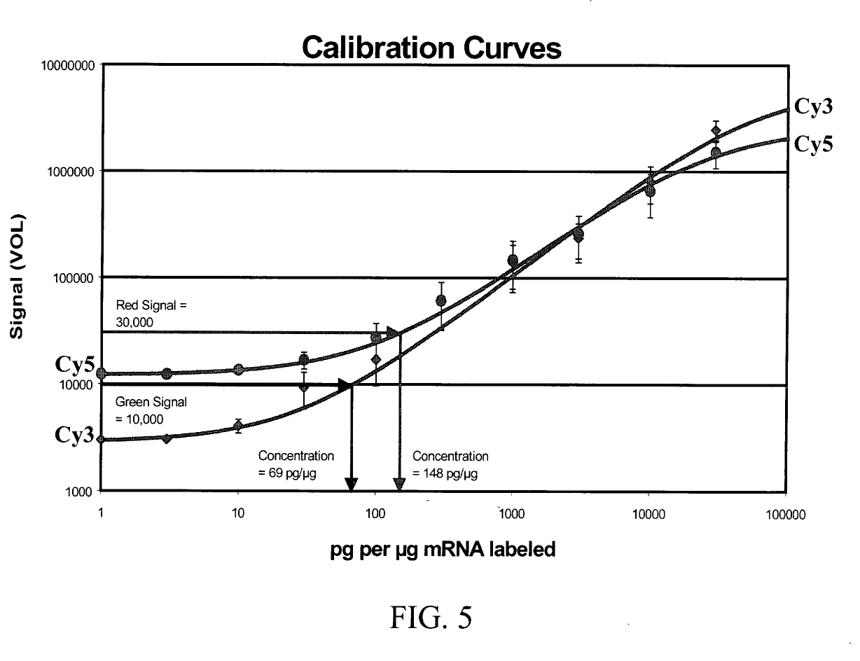

Spike mix performance was tested, including ratio performance and Calibration curves (FIGS. 4 and 5). The mRNA from the final set of clones have been pre-mixed at specific concentrations and ratios (see Table 1 above) so they can serve as the various controls when hybridized to their corresponding control DNA spotted on the arrays. Ten calibrators (those included in the labeling reaction at a ratio of 1:1), spanning a dynamic range of 4.5 orders of magnitude, are included as calibration controls. Eight ratio controls are included, at two expression levels (low and medium to high) and reversed with respect to the reference and test samples.

FIG. 4 shows a scatter plot of raw signals for the calibration and ratio controls from a two-color hybridization experiment. The Calibrators are accurately and precisely clustered at the 45-degree line and the ratios at their expected target values at high (labeled ‘H’) and low (labeled ‘L’) levels of expression.

FIG. 5 shows calibration curves based on the Calibration controls for a hybridization experiment. In this “standard curve”, the Cy3 and Cy5 signals from the calibration controls are plotted as a function of the amount of mRNA in the spike mix. The error bars represent the 95% confidence intervals for the mean value. From such curves, attributes such as the limit of detection, the linear dynamic range and the signal saturation limit can be assessed. The application of the universal controls for the generation of standard curves can be the first step towards true quantitation of expression levels from microarray experiments.

The controls as shown in Table 1 can be used as references for microarray validation and standardization across biological species and experimental platforms. These controls can be used to verify the accuracy and precision of gene expression ratios, and the sensitivity and dynamic range of the microarray system. Through the use of Calibration (standard) curves, these controls may allow reporting gene expression levels in consistent mass units, improving the comparisons of results across laboratories

The above examples illustrate specific aspects of the present invention and are not intended to limit the scope thereof in any respect and should not be so construed.

Those skilled in the art having the benefit of the teachings of the present invention as set forth above, can effect numerous modifications thereto. These modifications are to be construed as being encompassed within the scope of the present invention as set forth in the appended claims.

What is claimed is:

1. A control for use in a gene expression analysis system comprising:
(a) a known amount of at least one DNA selected from the group consisting of
(i) SEQ ID Nos: 1-23;
(ii) a degenerate variant of the sequence set forth in (i); and
(iii) a complement of the sequence set forth in (i) and (ii); or
(b) a known amount of at least one mRNA transcribed from the group consisting of
(i) SEQ ID NOs: 24-46;
(ii) a degenerate variant of the sequence set forth in (i); and
(iii) a complement of the sequence set forth in (i) and (ii).

2. A method of using a control as a negative control in a gene expression analysis system comprising:
adding a known amount of said control DNA of claim 1, to a gene expression analysis system as a control sample;
subjecting the sample to hybridization conditions in the absence of complementary labeled mRNA;
examining the control sample for the absence or presence of signal.

3. A method of using controls in a gene expression analysis system comprising:
adding a known amount of said control DNA of claim 1, to a gene expression analysis system as a control sample;
subjecting the sample to hybridization conditions in the presence of a known amount of labeled complementary mRNA of claim 1;
measuring the signal values for the labeled mRNA and determining the expression level of the DNA based on the measured signal value.

4. A method of using controls as calibrators in a gene expression analysis system comprising:
adding a known amount of a said control containing known amounts of several DNAs of claim 1, to a gene expression analysis system as control samples;
subjecting the samples to hybridization conditions in the presence of a said control containing known amounts of corresponding complementary labeled mRNAs of claim 1, each mRNA being at a different concentration;
measuring the signal values for the labeled mRNAs and constructing a dose-response or calibration curve based on the relationship between signal value and concentration of each mRNA.

5. A method of using controls as calibrators for gene expression ratios in a two-color gene expression analysis system comprising:
adding a known amount of at least one of said controls containing a known amount of DNA of claim 1, to a two-color gene expression analysis system as control samples;
subjecting the samples to hybridization conditions in the presence of a said control containing known amounts of two differently labeled corresponding complementary labeled mRNAs of claim 1, for each DNA sample present;
measuring the ratio of the signal values for the two differently labeled mRNAs and comparing the signal ratio to the ratio of concentrations of the two or more differently labeled mRNAs.


SEQUENCE LISTING (46 sequences)

SEQ ID NO: 1 - DNA, 936 bp, Saccharomyces cerevisiae
tgttgtccaagaaaggagggattttgttcatcagaaaagaattcagaaaagcaaggaaacagtactatcgtttagaatgtagaatgataggttgcttgctaattctattatggcacgaatgatacacccatattttcaacaaaatcaatacccactagcatcattgagccaactatttgtcaatgcaaccattaccggtacttcatcctgatttaacgagtctacttttttatcacgtcaaaatttacttgttttcctgtaaacccgaaataaaggcaaaaaagacctgggtgcaattacgaataaatgtacaataatcatcctgtttgcatagtaaacttccagttagagtcacacaacgcaatgaattttgacagttttctgtgcgatattctttggtaaacgtaaagaacaggcaacttttggtacaatggattctagcccatatggttcatttctggtgcattcgcaaagtcagtatttgtctagctgtgttttctggctgagagacattatgatgttattcattgttatggatatctctgtagctcatgctgcttatttctccctaaaaaagttttttctctcgaatacattcttgaccatttcatagtgaaattcttgtacttatttaaaaccaaaaatggaagtattcatacatccccctatcaaaaacactcaataagtttcgaattattcgttcgtctaaacagtgtccaatactcaaaggggtattcaagacggcacaaaatcagcatcttcccttatccgtgttccagaaataccacgctaaggtttttcctcctacaatccataaaatcattaaggaggcagcttgaaaaatcttgaaattcaaaagagttatcttgggctaatcgaaattaacgataaccagagtagaatattcaagatcacagctccaccttagtttcgagg

SEQ ID NO: 2 - DNA, 1000 bp, Saccharomyces cerevisiae
gcgaataaccaaaacgagactactttttaccattacaaccattttctttttccctatttctcactggttgacagaaatcagtgtgctatcatcctaccatatgcgctaaacttattgtctttctcctcctagagatgctgtattccatgcatattctgaacgatgggttggtgtttttatcaagcaaggttaatcacatggcgtggcttgctccacacatcagtagaaaacgcataccgcagcggaatccttaaataataagtgattttactgttcatcaactacaatcggactctttcacaattacccttcttgttttccacatttactgttaaatgaagggatgtacagaaggcttaggaaaacctgtgctgaatactggatggacactgcattcccacagtgaaacttttatagatacactgtcagttattttcgaactttcatcaagttgctgagttttagtatccctttgccttagctatatgtttgaatgagcaaaatatttgcaatgtctctagctttcttgaaatattggtttatattgagggcttggtaagatttcaaatttcactttgaaatactcaggagaaaaatcatgctcttttgataatttggtgactaaacatacataaaacagtttaattttgggtggtaatggctgtgtgactagctatagaaagaaaaaaattaaaaaaaaaaaaaaaaatcaagtagttcctgcactgcgacgtccattatagcattatgaattggtccctgatttacgcatgcgataaactatttttagcgcagccgcatattatccgagaataacttccgacataagaaaattcgcagaaaatagataaaaaactgctcttggcattcttcacttcctctattacacactgtgtcataccacaatcatctcacagtatgtatttgtatgtttatacatgctataacgtaaaacaatgtagaatatatatctaaatacctcacggttttagtttagtgc

SEQ ID NO: 3 - DNA, 980 bp, Saccharomyces cerevisiae
tgtgagaaattcactagcttcacctaaaagcaaaaaatctcaaaattcccaatattcacatagtctaaagtaccgatagcaaccaacatatataaacagtagtattttacgaagctgaattgcaagattagtgagaggagaataaccggataatttttttggattacgttattgttaaaggctataatattaggtgaaacagaatgtcctagaagtttttttctttcatgttaaatttattgattcttgcgcttcagcttttataaaacataagaactgtttcttcacgttaacttcttgtgccacatataatgatgtactagtaatatgggtactatttggcagatgatatttgatttttattcaagacggttactgtttctacgattgatattttcattcctggatatcatcttgccagatcacttacaatttaggccgcgcctgaattgaagagtacttcaatacgtagtgtactgtccaaactctcttccaaatttttaatatttagctggggttgggtaacaagtgagcaagggaaaaagtgaacattttaagaagaacaataaaatagcaagagatggaatggtaatgcttggctctcgagaagagtagcataaaacgagacttgtttaaaacaggatatgacatacttcaattcagctttccctatcagccgctcgagcagttatataggtgtgttgccggagtaatttggcggaggccaacagtggctaggcggcaacgcctggaacacgcgcttaaaagttctggaaggttcgcgaattgagaactgctcaggggcgaatacaggggcggccttggcggcaggggggaggcctctgtgaagttagttatataagacttgctgtcatcgtttttttgatcccggcaggaactatcttttattctcatacatacggtcaagaagtataattatacataacatagggacacgttcaggcaattgtccatatccc

SEQ ID NO: 4 - DNA, 937 bp, Saccharomyces cerevisiae
gatgtctgttttctttatgcaggatattaaatacaagtttgtgcttaagaaatttccatgaagatatcaacatttattgtaggaattgcataaatagatgaattatgtgccgctggacgtttatagatagcataagcacaatgacttaaaggttataatactcattgatatcactctgattataaaatcgtaatatgcgaataggtgaactaatcggaataacccatacgacacttcaagcttcaattctatttcaactgtagtgcctgctagtgaagaatacaaaagtagcatacgtgatgtgcaaaaaatgcgctacttatcacacaagtaccttgcgcaagaagggtactctaaaccggggccatcgcattaccagacggagatgtattctttatgaagcaataattggaggtgtatcaagttcgaaactgctgatgctatggatttacatctttcttatgcacaaggcttgcttgtgtttctgagtagttagtttttagatttttgtcaagtctggggtaagttaattcgagcaaaattaacggcacgttattctaatgcatatgttgttcatatattcttttacaaagaggtttggaatgatgtcaccgatgttagaatgttaggagaatttcatgtgaattttagtccaagtgttgaagttctcttctgcagttagggcacgtacatggcaacgatatcgtttttgatgtattaatcttagtaggcgttgagtttgtatgttacttttctcaggtgatgaagcgtgatgacgatgacaaaaatgggttataatagggcgcactatcatcatgcgtgattgatatttaaccaatgtcttgagtacatcaactccagaaaatgggtcattatatgcctagcatgtattatttgagacataaagttttatctcgagaccttgacgtataggaaa

SEQ ID NO: 5 - DNA, 940 bp, Saccharomyces cerevisiae
ctgtagatttgcatggacgcacgtcgcccatacgccaaactttggcaatgatactcgttattcgtaatatcagtccgtcaaggtgctgtgatttctctattttatattgcctattattttttcaaatgatttgagccgttttaaattgagtatgcaatgagtcttttgaatcaaccgtaaggcagttccataaccactgccacgaatacgtttcactaccttgaagaatctctaatgtaggccgtattcttcgcacttagttctgacgatgtagacatctcattatataagagcataagcgcctgtttctagaatcatttcttcgtgacccagctttttgagttatttcgcggtattttgaaacatttctcgagcttgacgtgaacatccttatatttcatgacaaactcgatcattggaacatccctgcctcgattttagagctagtatcaaatttcaatctctttgtgatggagccccgctcctatttcaaaagagaagtttcttgtatgcatatgttattgaagtctgattatagcaagtgcaatgtcgtctcaattattttaactatttttagccatacatgttagttatcctcaaagagagcctccagactgggaagcagtgtttgtcatttcaaataagtagatttcacagtttgtatgattttcgaagccaggattcattgggctttgagtaaagagaagccgcgtattacgaacagcttacgatattgtaaaatattcccttattgtggtgccccaatggatacatgccagagaaatgtctgtgaaattgaacaattacaatgacgagagcaagtaatccggcggccttgtctctctttcactagtaccgtctatatctcttgagcgccaatatgcgaaagctttcacaaggttgatgttcatggtattcggcgtcgatagcgaattgctta

SEQ ID NO: 6 - DNA, 950 bp, Saccharomyces cerevisiae
ttagtttggaacagcagtgtagataccgtccttggatagagcgctggagatagctggtctcaatctggtggagtaccatgggacaccagtgatgactctagtgacttgatcagcgggaataccagtcaacatagtggtgaaatcaccgtagttgaaaacagcttcagcaatttcaactgggtaagtttcagttggatgagcagcttggaacatatagtattcagccaaatgagctctgatatctgagacgtagacacctaattcgaccaggttaactctttcgtcagagggagataaagtagtggtggctggggcagcagcgacaccagcagcaatagcagcgacaccagcaacaattgaagttagtttgaccatttttttcgattgaacttttgtagatctttttagtgaagatgtgagctcactcgaatgtaaataacaatgccaaattgtcggaaagagttaatcaaagctgctctatttatatgccgttttttaataagcgacggacgaacagataaattgttgaatagctatttcactgctgatatttctcttacttgggctcccctatcccatactcttcaccactacaaatatgcagttgccctttcttcaacaatgctttttttatagatctcgtatacggatccgcgcctttgtactacctatatcttattatgatatatacaggagcacaggaatgttcggtacagggatgatacctttaaaggaagttttggcatgccttgacaacttcaattaatctttggccaagaaaatgaaccagaaatcaaattttattctgtgccctctgaacgagggcaatatccaatgtttgacactaaacggttgtcaggagaaaaattgaatgtttcccaaatcagaaacattaaaatccctctatatgatcagaggagtcgtacctgttagggtatgagcgaggaaac

SEQ ID NO: 7 - DNA, 982 bp, Saccharomyces cerevisiae
aatgagttaccgtctgttacttttgggacggtttttgcactaagaacagacgagtttacggttatcctcaacaagcaagcaagtatttgctaatctagatgccattccgaatcattactcatacgttactattgagagatgttttacaatagatgagaagaatacaatgtccagagctcctggtatgctagagtgcatattccaggtcttattcgaatcatatcataccgtccatttcaacaatggtgaaatgtggtccacatatatcagaaatcttaacatttagtgaggagagccagtagaaaaatgtgcgcaagcggaaagaagtcattcacagacacgtttaacaaaacaccaccacagcagctttgtctcttgattctgatcagtttgccatcgaagaagcaaaattgtggtgttatttttttcaaacaaaacttttttggcaacagcagttttcttctggatatttgtactttatcatccaaccgatgaaagctggtttcctgtcaacctacatttaaatggcccgtacttcttcaaaaccgctagataagcaaattaacccaacttttgagcgtcctaaattccccttggctcagaagactcgttaatatgggaagtttaagtcctaccatataatcaaattggaagctttctgtgttcgaatggctattctaaccgctgggctattaatcagaggggaagtgaaatgaccgagacgtattatacgtcatgttgacatcaacaatttaaggaaaaaaataaaaaaaagcaatgaaaaagggtttttttaagttgaagacccttttcaaatatatgttgctttgaattgtatctaccgtctcgtttcttctgctttaccgtttttttttgccttctttagatatgtcttttatgcttgaaaggtccggctttaatgcattcatctaaacgtagtattcctatttttgaactgctaccaatccaccatgactttact

SEQ ID NO: 8 - DNA, 905 bp, Saccharomyces cerevisiae
gtcaaactcccacagccaagtccaagagtacgaaaaaaaactttcacaacgagttcaaaaaatgtgggatgaagactcccgtttttcaccgcctagaaaacagcgtttgctgagaaaaaaaataaatcatcgagaagaagtatgtcatcataggatgttcccattgtaaggtgatgtgtaacatactcgaacaaagaatgtatagagctgaatatttctcctttaaatttcaaagaaaatgagaaggaaaatctcaaacagaaacttcgttctttttctcaagtaagcaaaagcttattgagacaaagcggaataactacgatattaataacgttgatgaagctcgaacaaagttagcgtcggttatgcttgcctatataaagatatatttgccttacattttcgttgaacgtagaatgatttttgcttttaataaattttttgttgttctttcagtgcttcttcaactttgatacgaaagcaagtgcattagtacaacaagaactggccacaactatactatactcattttttcttgcccgtgttttaaatgttttcatccacagcatttgatgggatgattggaagtgagacgttcgagaaaatccatattttgagtcaagaattcagataatatactgagatgattaggtatggctgggttctacaaaaacacaaatatccggctagcaatgatcactgagcaaattaaagcgttaactcactcattattgtagcttatgcgtttctcctcctctctttttttcctcgaaccggagtggaagatccaataacgtaatattactgatgttgttattaaagctggcaaaaataacatgaggcgtaaaaccgcactgcggtaagatgagggtataaggtggagatcaggcgaacaagctgttcta

SEQ ID NO: 9 - DNA, 908 bp, Saccharomyces cerevisiae
aacgatccaatgagcgtttcatgatgccattgtttaatcagagtgatgaaaaagaaatatttgcgaccttttttcgttacattgatcgtgaaattttaatcaaagataatataaggacgtgagatatttatctttttacttgaaattaacaatagaattgcgctaagcggaataagagctttcgtaaacctttctatttgcaccattgcgtcaacgtataaaatggtatgacctttacacaaacgcatgcttataatcttatgtttttcatagggtgtaatttggttgatgacgtagtctaaatttgatgctatctgcaattgaggtacatataagaggtcaatttcgggaccaacccttttaatcgaaaaaaacgtaattcactagggcaagggagaacttagcagctaatatcgtaaacctttcatactaaaaaaatgcacttaccatcaacaaaaaactcaggaccaatttccaagcttttctaggtgattgcctataacacaaaaagattcgctcatacatgagatttttacatgtaatagcaatttgttccgatcagttgaaggtcatcaacgcacggcaggtacatccacacctatcacaaagcccttcaataattcacctacgtaaagttataccgaaacatgcaaaatccatgaaaaattctgtatgataacgatcatatccttttgtattggtggtacgatgctcaaagatagttattgttgcacctgaggcaaaagcggaaatgaaaaatccagatggggccaaaagcagaagtattgtgtacaacaattgcttcagcagtttaccaaaccgtttcccagcaatcatcaaaagttgctttagccacatttccgcaagatatctttgtggctcaacgaagagggctattccaaatgcaa

SEQ ID NO: 10 - DNA, 909 bp, Saccharomyces cerevisiae
aactttcccccgtttaccacattgaagctgggtgtggaagatttatttgaagaaactaaaacgtaccctgtcatttcctgagtcccctttcaacttagtgtgaaagccgaacaattataatcctcggtagacaacagatttattgtactaaagttactcttcctgttatcttccttgattttactgttatagcaatgacccaccgcaatcaggagagccgccgtatggaatagcataccaagtcataaaatcgtcaacctattaacggggttcaggttctttttcagcgtagtagccctttaacaagcgctgacaaagttgacactcagagaaaattcaggatttattgtaatccagctactcatccttagatccgcttgcaggcatggtttttttcaccttgagaggctattttgggtaagccaggaaggctgaaaaatcccaaaaggacacagtaataagaaattgttgttgttgtatgatgcatttagaactcaaaagacgagtttctgaaaatgcttacaatactccataggtaacatgatttttttattaaaaaagtatactgttcctttgggtaaaaattatgcaacccttgagtgtccgatgaagataagactacgaaacaatttgcggtaaattttttctgctattgacatttacacatgctccaatccattaccctttccattctcgtaataaaacctcgaactgttatttcatatttacatctagacgggtatcggcctcaacaactccaaacaaaagtaaatagaaaagagccagacctatcgcaccgggtagagccagaaaatattttaaactatagttgacgtattctacggctgttgtttaggacaatactttttccttcacaggcttcgaattacgcacatgcagaactcctgt

SEQ ID NO: 11 - DNA, 929 bp, Saccharomyces cerevisiae
gtgagacctccggaattttgacgctgcaagtcaatctacgggaaagaagaaattttttaaacctaatgcaaaataagcttttcttggaaaataagattttcggcaataaaaggtaaatgcagccaaaaatcaaaatacttcagaagaagtcgtagcgaggactgctaaggggaagcggatttgaagatcctttccagaacaagaaggagccgaaagctgtcaggaactgttcctgattttttaggaaaacaattaataggtatctcgtctagagtagtatctcgagcttccagaagttgcagataatcaaaatcattgttttatccctttttttagattacagcttagaagagtagagagcaagtttactgaaacggttccttgtttacaataatattcctaacaaactttacgaattaggatgcagcatgattttttatattgcttcacttcctaaagtatgaatttttatccgtagtcgcaaacaaaacagctactggaaatctgcagcttgttaaaaaccggtagtttccgaatactcctcgtccttgagttgtataccgttaaacttcctagggtgtcatgtgtctggcccaattggcccacaaaatctggtcctattgacggttttcttttgattttcagcatcttcctctaagaaggacagaaaattatgtaatatatgggagaaacggcctcccaactgctaagtgtccccggcagcacgagtaagcaaaattcaggcaaactattgcattaagaagccgtacataattcagcgtgatatgatgaaattttgttaattgcaaattttagtacgatttggttgttagtgtgtgtttatgcaagtaattattgaaccctaagtagttactgtcttcttttgctgtaattcgtggattcacggccctccagcaacatggattgaa

SEQ ID NO: 12 - DNA, 900 bp, Saccharomyces cerevisiae
agtagcatttatgaccaaaagcgtacttaaattagcagcaaaaaaatttttaataacgaaactataaggaaaatacgaggtactgattatgagagtccccgtttctcatttttgagacatgatctgaacaaggctgaaaacagcaatctttttcgataacttttgcaaaaatttcaaacattgttgtttgaatgcagccaatttttatagggtacagagcttaatgctttacatgtgctttattttcggtactttccttaaagtgtctacattatctctcaggacttgaatgtcttcggctgaattactataaaatcttgagttttctctgaagtttaatcctaagacaatagtggtgagtgatgtagttcacgtgtgtgccactggtaataatagagataactatctcagttaagtttgaaaaggtaaaaaatagtttaagtagtcattttttgcgacggtcattcttctctgatgcacgttctttagactacctataaacaccattcttacggaattataatggaaataaaacatcagtacgtgttgctgtcggtgatagaggggtaacagaaccttaattgaaaaattagcacagtgcataatttattaacatgattgttttctgtgggaaataagaaatttcagcaccagtaaaagacgagaaatatagggcacataaatgcgctcttactcgtatgttccaggatgaaaatgtttagggcatcaagtattgccgaaagggcaatatgctttaacaccagaaaatccactgtatactcgttacgggtaaacaaagcaaaacgcagtgcgtgataatgtttctaaaatctctgcacactgttgaaatgcggctctgatactttagcccttagtacctgacggtgcctaaaatgagga

SEQ ID NO: 13 - DNA, 951 bp, Saccharomyces cerevisiae
tagttggaggttggtgagtaccagattgcttacaaaagaatagcgagccaacatttgctctgcctcaggcctcttggtgctgcttgaagactcatcttatatggcttttgtatgtcatgatttgttcttgtacattatgtgttgatattaaacaaattgatttttttttttttgcgatagcaagcagataatgaaagagacaaggacttggaacatccgataagactgcgccgatatcgatcttacagtccttcccttgtgtcatgactttcggaaaagcatcctcgtcgactggtagtttgctgtctgtcacgtgctgaagggtctgatacatttttttaaagataagagacggggtttacccttcggaggactaagcgagatctccaagtaaagatctcgcttatcaagaaagcagccaagtgtggaacgtccttttttttggtttcaaaaagatattcaacagtttacactgcagctttaattgcctcaaaaggatatcatgaggtgatctagggtcagaagggaaagattacagcatcttgagttgaatcacatctgcaaaaggtggtattattgacgttgctcttccttaatggaaactcatggggtttggaaaggaggtgcggtaatctatttttttcgaacacaaaacctaaccttgaaaagaaactgtccaatttcattgaacttacctcagaacgggccggagtctttgctttcagtctaacatggtctaatttcttcgaaaagcttcatttaattgttagactgtggttttacaaggaaaaaaccagtgctatactgaagcgatacccagaactaattaccttgtgtgacgattcggctcagcgaaacggacatggtaaaattgggaatttgaaagcaggcagcagccttgtacagcgacatgacgataggtttagaatccccatcacgtacgagttgaag

SEQ ID NO: 14 - DNA, 1010 bp, Saccharomyces cerevisiae
tcctagagtagcgattccccttcgcgtattcttacatcttcgaagagaacttctggtgtaagtataataaatattatagctctatcgaatggtgcaattatttaccaaattctcaataggaatccataatactacatacgatactaatattctagtatttttatacttattatttcttttttattacaccagcaatcgttgcaaattatcttctgatagaatttctgagggtatcctaaacttatgccattttcttggactgtaaatcatacttggatgttgtgcattagtcaataatcggttcttgttccaacgattacatgtaaatgaagggagaaataattatggtaaatcatgcggcggtccttttggtgatgcagtatccatagtcactacataacaatcttagtcaccttgtattgattcaccacataatcctgcagagcccgctatgtccttaatctgcgcgataactctcctacccctgaattttgagagcgccatagcaaaccgataaagctggcacaattaaaggtatcggtgttgtcagaattaggtgcctcctgcttttttttttttcctgctcttatatccgttatatccgaatgatttttatcgcttgtttaaaaaatactttcccgatatatatatatagtctccctttaaatttgtttccggtaagtttttaacaccaataaatgaaaagaaatgactacggtgatgaatatgagccgcgcattgaatcaggttatgtaagtatcagaacccctaattatgatgtcactcttacccttcgatggctaagcggcgactgggatgccgggaaaagctctacaaatctactaaaaaagtcaaatatacagctgtaaacttctttcctcgtctacatcatggtaacgattgttcaatctttacttcgtgtctttttttttttctatgtactttctattccaacctatgtgaagactaaaattcaccttagtaaacgtaaagacaatgacgataggtgc

SEQ ID NO: 15 - DNA, 945 bp, Saccharomyces cerevisiae
acaattgcatctgtgcttggctctcaagaacggtgtttggtgcatcaaaagttttcgactgcttatttggtcggaaatataaaaactcgatcctcttatctaagcagtatacattcttctttttgaaatgaatgtactccgtaatatcttcttatttggcattttcatccttaacttttgcatggctctgaactagtcagatagttgcccttttcagcaaacctcttattattgaaagcatggtgtacatccgttatactattatattataagaaattgggatgccaatttttttgcttttgttttgcctgttttccttcttttcgcaaaagtaattgcagatttaatagcaggatattataccgttggtaaaacttaaggattttatgaacaatagcttcaagtacagcattcatagaaccaactactaaggatgaaactagtatgtttttgtcaaaatattttcttgaccttgctgtaacatcaagatctgtttctctaagatattaaagttgagtaaaaacaaagctgatatgagaaaaatacgtaattgctccacataatacgtgggtcagacataaaggtagaatacttgatacagaagagattattcggtactcttgatggcgtgcttgaactggtgcctcttaacaaccggtaatatagtcagatgagtcactacgagtgtgtgtagtagcaagtgttttacctacgtggcagtaagagtagctctatggttgtgtaatagtggtgcttattcctaatgctctgaagtctgaagcggtacagttggtctggtctatatcatggtcaaaggagcaaacatatcttctgaagtgaccgcaaatagtactatgatgtggttggcaatataacttaaaaggaaataaccacaaggaattgcacccatgtacacagtttttcccggaaattgggaaaccagta

SEQ ID NO: 16 - DNA, 909 bp, Saccharomyces cerevisiae
gcataatgcccgctataaaccttattttttatatgggggtctggcgcttcgggaaaagagaggaaaacttgtaactcaatatatctcgatacaacattacgttttgtaaatttatcacaaaagccaaatgatgatatctctcttgcaagttatcgaacattgattggtaatttgtttgaaaattgttaatttattgaatatttcttttgcaaaagaaatagtctcagcgaaagctggttacaaaatttacatcatgagtttacgggatttgtaaatacgctttttgcataaaaatactttgccgtttcccacccttgcatattcacttactccccccttcatatactctatgtaatgatgattaagctttggccgctaagtctctcaattagtgttgattttggttttattcatatgattcttctttagtgaagtattgatcaattacgtgagtcagctttttgaaaaccccatttggaaggaattaggaaattattttgcttactacgaccactaatttaccgccatttctgggcctttttattgactattttgaccatgtgctcgactagaagaacggcatcataatctgctggtagagttagtctataatgattgttgaaaataaaggcataagagatattccacctaaaattcaagttattgactttattatcaggatcttagtatccttttttggtaagtcatattcaatgaactaggtctcgcaaactttttgttcgaaaagcggtagtgcatagttatgctaactctggatatatggcataaaccgtacaacactagcccatttttttggaagtagtgagggcagctagactgtatgatgaatattcgcctgcatactgagttttttggtccttttttttatgtggctggccttacgatatg

SEQ ID NO: 17 - DNA, 944 bp, Saccharomyces cerevisiae
gtttagtgcacccagactcgaatcttaaataccactttacacacctactaaattttgtcctcacaaaatgaagacaggattcaaaaccgattaatagtagcagaaactaaaaaagtacgaatattagtaaaattcatgttcttgaatcgagctactatctttgtcgggagggtaaacgattataactcaaaatgactggaactggtgattattaatttttacgtttcctgtgccaataagcggaagataagaggatagaagaaaagaaaggcggcacttggcgaactacaatggcgattatattcatggcgattatattcatacaaaggtaatggaggcctcggataatggacaatattgagaaaatccttatgcttacttctcttaataaaaaatagacacagccatttattatgcgtaaaaaagattacccacttgtcttcgatgcgtgctgctgccaatcaaccttttgagcggaacttcgagctcgcaatgcgtctggaatgttgctagagacagtcttggttatctgtgacatgtgtttcgttcaggcgtgtgagcatcttcttgttcgatttcaaaattaccgccttgactcgtgaaactggataattcgttggcgttttcatataagtcgtctgatggcgaaaacttttcctttacttagcatacagcaaatatccccatttgacggatttttgaaaaatgagcccgctaacccagaatgaactgcattaccaagcatttatgtaaacgttccgccaccatctttggtaaggtatactattatgttctggatttaaggttgattcacaatttttcatcaccaaaatctggtggcatgcctagttgtctggtttcaggcaatttagccatcatagaaaagcatcctctgtcttgagttgagaaaatgttactcatagagccaaacaaataaaccctgg

SEQ ID NO: 18 - DNA, 903 bp, Saccharomyces cerevisiae
ttcttgccgttctcatttcctgcacagtttctttgattatgtttgcagaagaatttctttatcgtttagtctaaacaaagaattcgttgtaaagaatttgagagcggatcttgcattttttatttatcatgcttatgttttttctttgatgtaagaagaagcaagtaagatatgtgaatatcttatcactaattcaaataactaagagagctcacaacgacaatttgtgacagcatgcgaagcaaagagcagtgataccagtatctttcatccagtaataacatacgactgatgttatagttaaatgttacattttgagagacttcaacctctcgaaaccaagaggttggttttaactctggtgacttcaagaagggtgggtaccttttacaaagcttgagacgaagcaatagtcagtctctgtataacaaggagaccacctcattttccagtaactcttgaggcatgtcggatggtttgccttgaataaaccgcagtcattataatgaatggcctgtactttcaaaacagtctggaaacagaaatccattgctgaggtaccttttagtagcactttcgttagtgaaggtttaaggttagttcttatttactgcacaagagtttacatttaaccactctaatagtaactgttagagtggtttaactgttaggtgatctgttcattccatttttcgtgttgtatctcaagatgagatagcttagcgttgctacatacataaatctaaacatataaacacctgtgtaactcgttaacgtctgggcttccatgcttctaccatttagaatgatgtagaccatttattccaagaggataagcaccctctgtgattcaaaatgataataagtgttgacgacaagttactctcgcagaattgttgtcaa

SEQ ID NO: 19 - DNA, 973 bp, Saccharomyces cerevisiae
tgcaatttaaagagcgtacctgtaaataagaaggaagaacgttatgttattaatggactttttagtgtcatcgaattttatgtaatatataagaaggtagaataatttggcaggataatgtgttagcaaaggaggaaatcgaatacctttaaaagagaaaaaattttttagctgcttaaatttctgtgttataccacccgatagattttgagttatgctttctaattgatctgactgcgaacgttttctttatgccatctgaattgtcaggaacaaagaagaaaaagaaaagtttttaaaaaatctgtggtcgtgtgtgatgtacctttcctttacatgcattaatgcgctctgaaatgtggtacgatatccttacagagaatatattttctgtatatcgtgcaatgttgaataacctatgaaggaaagtacccatcgctcaaggtaagcattccaggagggtcgccagaaacttaaactagttttagcgacagatccgaaaattgatagagacattgaaaaaatcactactccgtcctttttagtgctttctcaatgcataattttggtgcacgactaaaaaattctagaacactatagttgcattttttgggccggaagaagaaaaacgcatgtaactttaatgtcaaataaagttttcacctagtaagcgcgatacaaaaaaaacacagaaatagccataggaaagtgaattttgtcagccgactaaaattaaggttagcttacaaagcagcaaaaaatttgacatcgcacggtattccctgaaaaaggagcaggcaggtgctgtatatttttttcggttcctgcctcttacatggcgtcggtgtatcttaaatactaaagtgagctgactacccttttgagtgccctatgtgacctctgatctcgaaagtaaacaagagatacctaatttcacagccacttttgttgcggacactgacgggatgtgttg

SEQ ID NO: 20 - DNA, 900 bp, Saccharomyces cerevisiae
caaaaataacagcaagaaaagcggaaagaccatcgcaaggtggaaaggattataatggcacagcaaagtcgcacagagcactacagtatagcatagagtgctaatgagttgataggcccaattttgattatgccttctttttcatacacgacgccagaggacattattacattacagtagttcgccgctagatgacaaacgacatccttaccgatatgagatgtgcaaagctacataatggcaacaagcgttatgaacagccttgtctttacgaccacagaaaagccgtattagagctcttcagctgcaaaattttcttctaatatgatgcaaagccatcaaaaatcatgcatagttatgaaatacctgatgaaacgcttcgagttcgtgctcaagaaattactgaaaggttaccgagaagaaaaatatctatgagacacgataaggccccttctgaatccattgtcctgggcttgttcattctatttaccacttaaaattgatcctttcaaaggaatttttttctatttccaatagtatatttgtacaaaaactacaaaaatggataaaaaataacagtaatttgtgactactgtaaatatcactgatttggattttgtaatgagtactgctcatgcccatgccgatgcaagtggatcataaattttactaaacgatattcgataatgcgccaagcctttataaggaactcaaaataacccatatggacagtttcagaaggccaaataacgatcaaggacattcactcatgtttttcaaaggcgaagagtgtaaaattttcttctatatagttcgaatattttatcttataaatttcagtcgtcattttccacattcgaactcaaataatgataaagaacgctgcagtaatggcttaaa

SEQ ID NO: 21 - DNA, 950 bp, Saccharomyces cerevisiae
gcaagtatggggtagcaagctgcttaaagcttcttatcacactgtaacctctgttaaagcagctgcgttgttcttgcactctgcacgtaaatgacgacggccaatgtaaaaatagcagtcactgcaggcctcttggattgtacccgtaattacagcaattgccattagatttacgaagtcatttaattaaatggtttgatttatctccttaaatgcttccagaaatgggactagacttttttcactcaaacctgttcacaattattgctctttttcaattataaggtaaacaaggccatctatcagcaacacagtgctcgcattttttaattaaactatataaaaccaactatttgtggttgcgacttcactttttgttgaattactacccaatcattaatattgaagatgtgagatcatagatttattggctttgggcatctcaaatcccaagaggtcattttaaccaacaacattttaaaaagtagatttgtctgcctcagctatgagatgcgcatgtccctagcatctcatatctggttatattattttttccacttggtgaatgttgaaaaaaacaccactcgtccaatttatcagtttgcaggtctaatgtccttccctgttatttaaactgtatattgtaagcatgtcttatcgaaacaacttactcagttgtccgaaaacaaaactgcaaattctgtgtgtattcacgtactagaatcctgtcaaattggatcttgatttaagcttttatagcaacgaactttgcatactaagttttttttgttaaccggaactgccaagaagcattcagtaaaatacatcttcatcatttactgataatactcattcagactcatatcatactatttcgaattcattatacatcctcaaaaaccatattcttcagttgtaataaaagatagagcctgcatttgattcgattt

SEQ ID NO: 22 - DNA, 967 bp, Saccharomyces cerevisiae
gatttaatacagtacctttcttcgctaggatctatatgcgaatatatcacatatgtaaattataagctcatcgcaaaaccaaaaaaaaaaaaattttcaataatttttcactaatcttcaaaaacaaatggggtaacccgtacaagagttattaaaacccaaaatgacaaaatcgcgacaattcaatcctacttaattagcaataacatactagcggtagagctactatcacatgttgaaccttgaatgctcaattcattgtactcaatactgctatcaaaagaaaaaaaatgtattaattatattcttgtcaaaatcaattttacactataagaggaaaatgttcttcagtcctagtaacattagttttctccctttgctagagactttacataatatcctagaaggtaaaattcgataatacagcagtaaagtcgtatattggtagcaatccttggtgacgctgactttttttttttgtaattttattgtttagttcatgataaaaaacttcaaatcacttttaatctggtagacagagaaaacaaatcgaaacgaaaatagagaactacgaataaaaaaatataagtggagaagatcgtcactacgcattaaacaatattgatcgctcaatgccagtactgcgcgtaaaagtttagtaacttaacgatttaggcacaatttgagaaaaatttcgccctgcagtaagtatgttattcagtacgatataaagctgaggttttatgctggcaacgttcagattttttaggttatcagcaatgttaaaatattaaataggatacttttattgtttgagaccaccctcaatgccagatatgttaaacgcttttttctggagtgaggtatcatagaaaaaggctcgagtacatcaagcacttaaaggttcaacactctactgttacttctttaagctaagctattcatacataatagtccatcaaagtgg

SEQ ID NO: 23 - DNA, 981 bp, Saccharomyces cerevisiae
gcaatttgcagttcaacttttcaatgatgatttagaatgatctaacactggaagttgaagtttttcaaaaatttgctgtaaaatttgaccgatttgtgagattcttcctggctgtcagaatatggggccgtagtatattgtcagacctgttcctttaagaggtgatggtgataggcgttgagtatgtgtagtgtttgacccgagggtatggttttcacaagtactgcgcactgtattgtgaaagcagcttcggggtgcgtgattaaaaaatgcgaccaagaataaacaggtacatcataacaagggccatttgaattgcatttatcaggatttgtaaccttgttctaaagaggcatcgtatagtttaagttcattttccacccaatttgatgacggtgtggaccttaacctattgtcttgaaatttaggttatctcttagatatcacatgtgattaccccagtgaacgcgtataagcttacagaaaggaaaaccggttggctcagtcaaaactgttgcagatttgggctcccctgaatatttgagacatccctaaaatgaagagatatatacagctaattttgaatgaaaatttaaaattcgcaatgaacagtactagagatgagcttttgaagtcctttcaaattatttgttcttccagttgatattttttattttatataccagtaccaaatataatcttgccatacatttacctttttgaggttgttcaacggaaatccagtgtatttacacattcttggaaacccatcgcttataatacgaactaatttatttatgaacaaaggctttggaaaagtatccctactttttacgacgctaaatcatgatacgaaactttaggaagattaacagtcactccataaaatcagaaagtattcgctaatagtggaaagaaatggttatataaagatggaaatatcttgaaagagacagtttaacccgaagttctgtcaaagtg

SEQ ID NO: 24 - DNA, 807 bp, Saccharomyces cerevisiae
ttcagaaaagcaaggaaacagtactatcgtttagaatgtagaatgataggttgcttgctaattctattatggcacgaatgatacacccatattttcaacaaaatcaatacccactagcatcattgagccaactatttgtcaatgcaaccattaccggtacttcatcctgatttaacgagtctacttttttatcacgtcaaaatttacttgttttcctgtaaacccgaaataaaggcaaaaaagacctgggtgcaattacgaataaatgtacaataatcatcctgtttgcatagtaaacttccagttagagtcacacaacgcaatgaattttgacagttttctgtgcgatattctttggtaaacgtaaagaacaggcaacttttggtacaatggattctagcccatatggttcatttctggtgcattcgcaaagtcagtatttgtctagctgtgttttctggctgagagacattatgatgttattcattgttatggatatctctgtagctcatgctgcttatttctccctaaaaaagttttttctctcgaatacattcttgaccatttcatagtgaaattcttgtacttatttaaaaccaaaaatggaagtattcatacatccccctatcaaaaacactcaataagtttcgaattattcgttcgtctaaacagtgtccaatactcaaaggggtattcaagacggcacaaaatcagcatcttcccttatccgtgttccagaaataccacgctaaggtttttcctcctacaatccataaaatcattaaggaggcagcttgaaaaatcttg

SEQ ID NO: 25 - DNA, 762 bp, Saccharomyces cerevisiae
tttctttttccctatttctcactggttgacagaaatcagtgtgctatcatcctaccatatgcgctaaacttattgtctttctcctcctagagatgctgtattccatgcatattctgaacgatgggttggtgtttttatcaagcaaggttaatcacatggcgtggcttgctccacacatcagtagaaaacgcataccgcagcggaatccttaaataataagtgattttactgttcatcaactacaatcggactctttcacaattacccttcttgttttccacatttactgttaaatgaagggatgtacagaaggcttaggaaaacctgtgctgaatactggatggacactgcattcccacagtgaaacttttatagatacactgtcagttattttcgaactttcatcaagttgctgagttttagtatccctttgccttagctatatgtttgaatgagcaaaatatttgcaatgtctctagctttcttgaaatattggtttatattgagggcttggtaagatttcaaatttcactttgaaatactcaggagaaaaatcatgctcttttgataatttggtgactaaacatacataaaacagtttaattttgggtggtaatggctgtgtgactagctatagaaagaaaaaaattaaaaaaaaaaaaaaaaatcaagtagttcctgcactgcgacgtccattatagcattatgaattggtccctgatttacgcatgcgataaactatttttagcgcagccgcatatt

SEQ ID NO: 26 - DNA, 874 bp, Saccharomyces cerevisiae
aaaatctcaaaattcccaatattcacatagtctaaagtaccgatagcaaccaacatatataaacagtagtattttacgaagctgaattgcaagattagtgagaggagaataaccggataatttttttggattacgttattgttaaaggctataatattaggtgaaacagaatgtcctagaagtttttttctttcatgttaaatttattgattcttgcgcttcagcttttataaaacataagaactgtttcttcacgttaacttcttgtgccacatataatgatgtactagtaatatgggtactatttggcagatgatatttgatttttattcaagacggttactgtttctacgattgatattttcattcctggatatcatcttgccagatcacttacaatttaggccgcgcctgaattgaagagtacttcaatacgtagtgtactgtccaaactctcttccaaatttttaatatttagctggggttgggtaacaagtgagcaagggaaaaagtgaacattttaagaagaacaataaaatagcaagagatggaatggtaatgcttggctctcgagaagagtagcataaaacgagacttgtttaaaacaggatatgacatacttcaattcagctttccctatcagccgctcgagcagttatataggtgtgttgccggagtaatttggcggaggccaacagtggctaggcggcaacgcctggaacacgcgcttaaaagttctggaaggttcgcgaattgagaactgctcaggggcgaatacaggggcggccttggcggcaggggggaggcctctgtgaagttagttatataagacttgctgtcatcgtttttttgatcccggcaggaactatctttt

SEQ ID NO: 27 - DNA, 797 bp, Saccharomyces cerevisiae
aatagatgaattatgtgccgctggacgtttatagatagcataagcacaatgacttaaaggttataatactcattgatatcactctgattataaaatcgtaatatgcgaataggtgaactaatcggaataacccatacgacacttcaagcttcaattctatttcaactgtagtgcctgctagtgaagaatacaaaagtagcatacgtgatgtgcaaaaaatgcgctacttatcacacaagtaccttgcgcaagaagggtactctaaaccggggccatcgcattaccagacggagatgtattctttatgaagcaataattggaggtgtatcaagttcgaaactgctgatgctatggatttacatctttcttatgcacaaggcttgcttgtgtttctgagtagttagtttttagatttttgtcaagtctggggtaagttaattcgagcaaaattaacggcacgttattctaatgcatatgttgttcatatattcttttacaaagaggtttggaatgatgtcaccgatgttagaatgttaggagaatttcatgtgaattttagtccaagtgttgaagttctcttctgcagttagggcacgtacatggcaacgatatcgtttttgatgtattaatcttagtaggcgttgagtttgtatgttacttttctcaggtgatgaagcgtgatgacgatgacaaaaatgggttataatagggcgcactatcatcatgcgtgattgatatttaaccaatgtcttgagtacatcaactccagaaaatgggtcattatatgcctagcatgt

SEQ ID NO: 28 - DNA, 808 bp, Saccharomyces cerevisiae
tggcaatgatactcgttattcgtaatatcagtccgtcaaggtgctgtgatttctctattttatattgcctattattttttcaaatgatttgagccgttttaaattgagtatgcaatgagtcttttgaatcaaccgtaaggcagttccataaccactgccacgaatacgtttcactaccttgaagaatctctaatgtaggccgtattcttcgcacttagttctgacgatgtagacatctcattatataagagcataagcgcctgtttctagaatcatttcttcgtgacccagctttttgagttatttcgcggtattttgaaacatttctcgagcttgacgtgaacatccttatatttcatgacaaactcgatcattggaacatccctgcctcgattttagagctagtatcaaatttcaatctctttgtgatggagccccgctcctatttcaaaagagaagtttcttgtatgcatatgttattgaagtctgattatagcaagtgcaatgtcgtctcaattattttaactatttttagccatacatgttagttatcctcaaagagagcctccagactgggaagcagtgtttgtcatttcaaataagtagatttcacagtttgtatgattttcgaagccaggattcattgggctttgagtaaagagaagccgcgtattacgaacagcttacgatattgtaaaatattcccttattgtggtgccccaatggatacatgccagagaaatgtctgtgaaattgaacaattacaatgacgagagcaagtaatccggcggccttgtctctctttcac

SEQ ID NO: 29 - DNA, 709 bp, Saccharomyces cerevisiae
agataccgtccttggatagagcgctggagatagctggtctcaatctggtggagtaccatgggacaccagtgatgactctagtgacttgatcagcgggaataccagtcaacatagtggtgaaatcaccgtagttgaaaacagcttcagcaatttcaactgggtaagtttcagttggatgagcagcttggaacatatagtattcagccaaatgagctctgatatctgagacgtagacacctaattcgaccaggttaactctttcgtcagagggagataaagtagtggtggctggggcagcagcgacaccagcagcaatagcagcgacaccagcaacaattgaagttagtttgaccatttttttcgattgaacttttgtagatctttttagtgaagatgtgagctcactcgaatgtaaataacaatgccaaattgtcggaaagagttaatcaaagctgctctatttatatgccgttttttaataagcgacggacgaacagataaattgttgaatagctatttcactgctgatatttctcttacttgggctcccctatcccatactcttcaccactacaaatatgcagttgccctttcttcaacaatgctttttttatagatctcgtatacggatccgcgcctttgtactacctatatcttattatgatatatacaggagcacaggaatgttcggtacagggatgataccttt

SEQ ID NO: 30 - DNA, 893 bp, Saccharomyces cerevisiae
ttgggacggtttttgcactaagaacagacgagtttacggttatcctcaacaagcaagcaagtatttgctaatctagatgccattccgaatcattactcatacgttactattgagagatgttttacaatagatgagaagaatacaatgtccagagctcctggtatgctagagtgcatattccaggtcttattcgaatcatatcataccgtccatttcaacaatggtgaaatgtggtccacatatatcagaaatcttaacatttagtgaggagagccagtagaaaaatgtgcgcaagcggaaagaagtcattcacagacacgtttaacaaaacaccaccacagcagctttgtctcttgattctgatcagtttgccatcgaagaagcaaaattgtggtgttatttttttcaaacaaaacttttttggcaacagcagttttcttctggatatttgtactttatcatccaaccgatgaaagctggtttcctgtcaacctacatttaaatggcccgtacttcttcaaaaccgctagataagcaaattaacccaacttttgagcgtcctaaattccccttggctcagaagactcgttaatatgggaagtttaagtcctaccatataatcaaattggaagctttctgtgttcgaatggctattctaaccgctgggctattaatcagaggggaagtgaaatgaccgagacgtattatacgtcatgttgacatcaacaatttaaggaaaaaaataaaaaaaagcaatgaaaaagggtttttttaagttgaagacccttttcaaatatatgttgctttgaattgtatctaccgtctcgtttcttctgctttaccgtttttttttgccttctttagatatgtcttttatgcttgaaaggtccggc

SEQ ID NO: 31 - DNA, 788 bp, Saccharomyces cerevisiae
ttcaccgcctagaaaacagcgtttgctgagaaaaaaaataaatcatcgagaagaagtatgtcatcataggatgttcccattgtaaggtgatgtgtaacatactcgaacaaagaatgtatagagctgaatatttctcctttaaatttcaaagaaaatgagaaggaaaatctcaaacagaaacttcgttctttttctcaagtaagcaaaagcttattgagacaaagcggaataactacgatattaataacgttgatgaagctcgaacaaagttagcgtcggttatgcttgcctatataaagatatatttgccttacattttcgttgaacgtagaatgatttttgcttttaataaattttttgttgttctttcagtgcttcttcaactttgatacgaaagcaagtgcattagtacaacaagaactggccacaactatactatactcattttttcttgcccgtgttttaaatgttttcatccacagcatttgatgggatgattggaagtgagacgttcgagaaaatccatattttgagtcaagaattcagataatatactgagatgattaggtatggctgggttctacaaaaacacaaatatccggctagcaatgatcactgagcaaattaaagcgttaactcactcattattgtagcttatgcgtttctcctcctctctttttttcctcgaaccggagtggaagatccaataacgtaatattactgatgttgttattaaagctggcaaaaataacatgaggcgtaaaaccgcactgcggtaagatgagggt

SEQ ID NO: 32 - DNA, 825 bp, Saccharomyces cerevisiae
tgaaaaagaaatatttgcgaccttttttcgttacattgatcgtgaaattttaatcaaagataatataaggacgtgagatatttatctttttacttgaaattaacaatagaattgcgctaagcggaataagagctttcgtaaacctttctatttgcaccattgcgtcaacgtataaaatggtatgacctttacacaaacgcatgcttataatcttatgtttttcatagggtgtaatttggttgatgacgtagtctaaatttgatgctatctgcaattgaggtacatataagaggtcaatttcgggaccaacccttttaatcgaaaaaaacgtaattcactagggcaagggagaacttagcagctaatatcgtaaacctttcatactaaaaaaatgcacttaccatcaacaaaaaactcaggaccaatttccaagcttttctaggtgattgcctataacacaaaaagattcgctcatacatgagatttttacatgtaatagcaatttgttccgatcagttgaaggtcatcaacgcacggcaggtacatccacacctatcacaaagcccttcaataattcacctacgtaaagttataccgaaacatgcaaaatccatgaaaaattctgtatgataacgatcatatccttttgtattggtggtacgatgctcaaagatagttattgttgcacctgaggcaaaagcggaaatgaaaaatccagatggggccaaaagcagaagtattgtgtacaacaattgcttcagcagtttaccaaaccgtttcccagcaatcatcaaaagttgctttagccacatttccgcaagatat

SEQ ID NO: 33 - DNA, 775 bp, Saccharomyces cerevisiae
attgaagctgggtgtggaagatttatttgaagaaactaaaacgtaccctgtcatttcctgagtcccctttcaacttagtgtgaaagccgaacaattataatcctcggtagacaacagatttattgtactaaagttactcttcctgttatcttccttgattttactgttatagcaatgacccaccgcaatcaggagagccgccgtatggaatagcataccaagtcataaaatcgtcaacctattaacggggttcaggttctttttcagcgtagtagccctttaacaagcgctgacaaagttgacactcagagaaaattcaggatttattgtaatccagctactcatccttagatccgcttgcaggcatggtttttttcaccttgagaggctattttgggtaagccaggaaggctgaaaaatcccaaaaggacacagtaataagaaattgttgttgttgtatgatgcatttagaactcaaaagacgagtttctgaaaatgcttacaatactccataggtaacatgatttttttattaaaaaagtatactgttcctttgggtaaaaattatgcaacccttgagtgtccgatgaagataagactacgaaacaatttgcggtaaattttttctgctattgacatttacacatgctccaatccattaccctttccattctcgtaataaaacctcgaactgttatttcatatttacatctagacgggtatcggcctcaacaactccaaacaaaagtaaatagaaaagagccagacctatcgc

SEQ ID NO: 34 - DNA, 884 bp, Saccharomyces cerevisiae
gctgcaagtcaatctacgggaaagaagaaattttttaaacctaatgcaaaataagcttttcttggaaaataagattttcggcaataaaaggtaaatgcagccaaaaatcaaaatacttcagaagaagtcgtagcgaggactgctaaggggaagcggatttgaagatcctttccagaacaagaaggagccgaaagctgtcaggaactgttcctgattttttaggaaaacaattaataggtatctcgtctagagtagtatctcgagcttccagaagttgcagataatcaaaatcattgttttatccctttttttagattacagcttagaagagtagagagcaagtttactgaaacggttccttgtttacaataatattcctaacaaactttacgaattaggatgcagcatgattttttatattgcttcacttcctaaagtatgaatttttatccgtagtcgcaaacaaaacagctactggaaatctgcagcttgttaaaaaccggtagtttccgaatactcctcgtccttgagttgtataccgttaaacttcctagggtgtcatgtgtctggcccaattggcccacaaaatctggtcctattgacggttttcttttgattttcagcatcttcctctaagaaggacagaaaattatgtaatatatgggagaaacggcctcccaactgctaagtgtccccggcagcacgagtaagcaaaattcaggcaaactattgcattaagaagccgtacataattcagcgtgatatgatgaaattttgttaattgcaaattttagtacgatttggttgttagtgtgtgtttatgcaagtaattattgaaccctaagtagttactgtcttcttttgctgtaattcgtggattcacg

SEQ ID NO: 35 - DNA, 776 bp, Saccharomyces cerevisiae
gtccccgtttctcatttttgagacatgatctgaacaaggctgaaaacagcaatctttttcgataacttttgcaaaaatttcaaacattgttgtttgaatgcagccaatttttatagggtacagagcttaatgctttacatgtgctttattttcggtactttccttaaagtgtctacattatctctcaggacttgaatgtcttcggctgaattactataaaatcttgagttttctctgaagtttaatcctaagacaatagtggtgagtgatgtagttcacgtgtgtgccactggtaataatagagataactatctcagttaagtttgaaaaggtaaaaaatagtttaagtagtcattttttgcgacggtcattcttctctgatgcacgttctttagactacctataaacaccattcttacggaattataatggaaataaaacatcagtacgtgttgctgtcggtgatagaggggtaacagaaccttaattgaaaaattagcacagtgcataatttattaacatgattgttttctgtgggaaataagaaatttcagcaccagtaaaagacgagaaatatagggcacataaatgcgctcttactcgtatgttccaggatgaaaatgtttagggcatcaagtattgccgaaagggcaatatgctttaacaccagaaaatccactgtatactcgttacgggtaaacaaagcaaaacgcagtgcgtgataatgtttctaaaatctctgcacactgttgaaatgcggctctgatactttagcc

SEQ ID NO: 36 - DNA, 714 bp, Saccharomyces cerevisiae
ccagattgcttacaaaagaatagcgagccaacatttgctctgcctcaggcctcttggtgctgcttgaagactcatcttatatggcttttgtatgtcatgatttgttcttgtacattatgtgttgatattaaacaaattgatttttttttttttgcgatagcaagcagataatgaaagagacaaggacttggaacatccgataagactgcgccgatatcgatcttacagtccttcccttgtgtcatgactttcggaaaagcatcctcgtcgactggtagtttgctgtctgtcacgtgctgaagggtctgatacatttttttaaagataagagacggggtttacccttcggaggactaagcgagatctccaagtaaagatctcgcttatcaagaaagcagccaagtgtggaacgtccttttttttggtttcaaaaagatattcaacagtttacactgcagctttaattgcctcaaaaggatatcatgaggtgatctagggtcagaagggaaagattacagcatcttgagttgaatcacatctgcaaaaggtggtattattgacgttgctcttccttaatggaaactcatggggtttggaaaggaggtgcggtaatctatttttttcgaacacaaaacctaaccttgaaaagaaactgtccaatttcattgaacttacctcagaacgggccggagtctttgctttcagtctaacatg

SEQ ID NO: 37 - DNA, 757 bp, Saccharomyces cerevisiae
ttcgcgtattcttacatcttcgaagagaacttctggtgtaagtataataaatattatagctctatcgaatggtgcaattatttaccaaattctcaataggaatccataatactacatacgatactaatattctagtatttttatacttattatttcttttttattacaccagcaatcgttgcaaattatcttctgatagaatttctgagggtatcctaaacttatgccattttcttggactgtaaatcatacttggatgttgtgcattagtcaataatcggttcttgttccaacgattacatgtaaatgaagggagaaataattatggtaaatcatgcggcggtccttttggtgatgcagtatccatagtcactacataacaatcttagtcaccttgtattgattcaccacataatcctgcagagcccgctatgtccttaatctgcgcgataactctcctacccctgaattttgagagcgccatagcaaaccgataaagctggcacaattaaaggtatcggtgttgtcagaattaggtgcctcctgcttttttttttttcctgctcttatatccgttatatccgaatgatttttatcgcttgtttaaaaaatactttcccgatatatatatatagtctccctttaaatttgtttccggtaagtttttaacaccaataaatgaaaagaaatgactacggtgatgaatatgagccgcgcattgaatcaggttatgtaagtatcagaacccctaattatg

SEQ ID NO: 38 - DNA, 891 bp, Saccharomyces cerevisiae
ctcaagaacggtgtttggtgcatcaaaagttttcgactgcttatttggtcggaaatataaaaactcgatcctcttatctaagcagtatacattcttctttttgaaatgaatgtactccgtaatatcttcttatttggcattttcatccttaacttttgcatggctctgaactagtcagatagttgcccttttcagcaaacctcttattattgaaagcatggtgtacatccgttatactattatattataagaaattgggatgccaatttttttgcttttgttttgcctgttttccttcttttcgcaaaagtaattgcagatttaatagcaggatattataccgttggtaaaacttaaggattttatgaacaatagcttcaagtacagcattcatagaaccaactactaaggatgaaactagtatgtttttgtcaaaatattttcttgaccttgctgtaacatcaagatctgtttctctaagatattaaagttgagtaaaaacaaagctgatatgagaaaaatacgtaattgctccacataatacgtgggtcagacataaaggtagaatacttgatacagaagagattattcggtactcttgatggcgtgcttgaactggtgcctcttaacaaccggtaatatagtcagatgagtcactacgagtgtgtgtagtagcaagtgttttacctacgtggcagtaagagtagctctatggttgtgtaatagtggtgcttattcctaatgctctgaagtctgaagcggtacagttggtctggtctatatcatggtcaaaggagcaaacatatcttctgaagtgaccgcaaatagtactatgatgtggttggcaatataacttaaaaggaaataaccacaaggaattgcacccatgta

SEQ ID NO: 39 - DNA, 848 bp, Saccharomyces cerevisiae
tttttatatgggggtctggcgcttcgggaaaagagaggaaaacttgtaactcaatatatctcgatacaacattacgttttgtaaatttatcacaaaagccaaatgatgatatctctcttgcaagttatcgaacattgattggtaatttgtttgaaaattgttaatttattgaatatttcttttgcaaaagaaatagtctcagcgaaagctggttacaaaatttacatcatgagtttacgggatttgtaaatacgctttttgcataaaaatactttgccgtttcccacccttgcatattcacttactccccccttcatatactctatgtaatgatgattaagctttggccgctaagtctctcaattagtgttgattttggttttattcatatgattcttctttagtgaagtattgatcaattacgtgagtcagctttttgaaaaccccatttggaaggaattaggaaattattttgcttactacgaccactaatttaccgccatttctgggcctttttattgactattttgaccatgtgctcgactagaagaacggcatcataatctgctggtagagttagtctataatgattgttgaaaataaaggcataagagatattccacctaaaattcaagttattgactttattatcaggatcttagtatccttttttggtaagtcatattcaatgaactaggtctcgcaaactttttgttcgaaaagcggtagtgcatagttatgctaactctggatatatggcataaaccgtacaacactagcccatttttttggaagtagtgagggcagctagactgtatgatgaatattcgcctgcatactgagttttt

SEQ ID NO: 40 - DNA, 806 bp, Saccharomyces cerevisiae
tgaagacaggattcaaaaccgattaatagtagcagaaactaaaaaagtacgaatattagtaaaattcatgttcttgaatcgagctactatctttgtcgggagggtaaacgattataactcaaaatgactggaactggtgattattaatttttacgtttcctgtgccaataagcggaagataagaggatagaagaaaagaaaggcggcacttggcgaactacaatggcgattatattcatggcgattatattcatacaaaggtaatggaggcctcggataatggacaatattgagaaaatccttatgcttacttctcttaataaaaaatagacacagccatttattatgcgtaaaaaagattacccacttgtcttcgatgcgtgctgctgccaatcaaccttttgagcggaacttcgagctcgcaatgcgtctggaatgttgctagagacagtcttggttatctgtgacatgtgtttcgttcaggcgtgtgagcatcttcttgttcgatttcaaaattaccgccttgactcgtgaaactggataattcgttggcgttttcatataagtcgtctgatggcgaaaacttttcctttacttagcatacagcaaatatccccatttgacggatttttgaaaaatgagcccgctaacccagaatgaactgcattaccaagcatttatgtaaacgttccgccaccatctttggtaaggtatactattatgttctggatttaaggttgattcacaatttttcatcaccaaaatctggtggcatgcctagttgtctggtttcaggcaatttagcc

SEQ ID NO: 41 - DNA, 763 bp, Saccharomyces cerevisiae
aatttgagagcggatcttgcattttttatttatcatgcttatgttttttctttgatgtaagaagaagcaagtaagatatgtgaatatcttatcactaattcaaataactaagagagctcacaacgacaatttgtgacagcatgcgaagcaaagagcagtgataccagtatctttcatccagtaataacatacgactgatgttatagttaaatgttacattttgagagacttcaacctctcgaaaccaagaggttggttttaactctggtgacttcaagaagggtgggtaccttttacaaagcttgagacgaagcaatagtcagtctctgtataacaaggagaccacctcattttccagtaactcttgaggcatgtcggatggtttgccttgaataaaccgcagtcattataatgaatggcctgtactttcaaaacagtctggaaacagaaatccattgctgaggtaccttttagtagcactttcgttagtgaaggtttaaggttagttcttatttactgcacaagagtttacatttaaccactctaatagtaactgttagagtggtttaactgttaggtgatctgttcattccatttttcgtgttgtatctcaagatgagatagcttagcgttgctacatacataaatctaaacatataaacacctgtgtaactcgttaacgtctgggcttccatgcttctaccatttagaatgatgtagaccatttattccaagaggataagcaccctctgtgattcaaaat

SEQ ID NO: 42 - DNA, 873 bp, Saccharomyces cerevisiae
ttaatggactttttagtgtcatcgaattttatgtaatatataagaaggtagaataatttggcaggataatgtgttagcaaaggaggaaatcgaatacctttaaaagagaaaaaattttttagctgcttaaatttctgtgttataccacccgatagattttgagttatgctttctaattgatctgactgcgaacgttttctttatgccatctgaattgtcaggaacaaagaagaaaaagaaaagtttttaaaaaatctgtggtcgtgtgtgatgtacctttcctttacatgcattaatgcgctctgaaatgtggtacgatatccttacagagaatatattttctgtatatcgtgcaatgttgaataacctatgaaggaaagtacccatcgctcaaggtaagcattccaggagggtcgccagaaacttaaactagttttagcgacagatccgaaaattgatagagacattgaaaaaatcactactccgtcctttttagtgctttctcaatgcataattttggtgcacgactaaaaaattctagaacactatagttgcattttttgggccggaagaagaaaaacgcatgtaactttaatgtcaaataaagttttcacctagtaagcgcgatacaaaaaaaacacagaaatagccataggaaagtgaattttgtcagccgactaaaattaaggttagcttacaaagcagcaaaaaatttgacatcgcacggtattccctgaaaaaggagcaggcaggtgctgtatatttttttcggttcctgcctcttacatggcgtcggtgtatcttaaatactaaagtgagctgactacccttttgagtgccctatgtgacctctgatctcgaaagtaaacaaga

SEQ ID NO: 43 - DNA, 800 bp, Saccharomyces cerevisiae
ttataatggcacagcaaagtcgcacagagcactacagtatagcatagagtgctaatgagttgataggcccaattttgattatgccttctttttcatacacgacgccagaggacattattacattacagtagttcgccgctagatgacaaacgacatccttaccgatatgagatgtgcaaagctacataatggcaacaagcgttatgaacagccttgtctttacgaccacagaaaagccgtattagagctcttcagctgcaaaattttcttctaatatgatgcaaagccatcaaaaatcatgcatagttatgaaatacctgatgaaacgcttcgagttcgtgctcaagaaattactgaaaggttaccgagaagaaaaatatctatgagacacgataaggccccttctgaatccattgtcctgggcttgttcattctatttaccacttaaaattgatcctttcaaaggaatttttttctatttccaatagtatatttgtacaaaaactacaaaaatggataaaaaataacagtaatttgtgactactgtaaatatcactgatttggattttgtaatgagtactgctcatgcccatgccgatgcaagtggatcataaattttactaaacgatattcgataatgcgccaagcctttataaggaactcaaaataacccatatggacagtttcagaaggccaaataacgatcaaggacattcactcatgtttttcaaaggcgaagagtgtaaaattttcttctatatagttcgaatattttatcttataaatttcagtcgtcattt

SEQ ID NO: 44 - DNA, 850 bp, Saccharomyces cerevisiae
tctgttaaagcagctgcgttgttcttgcactctgcacgtaaatgacgacggccaatgtaaaaatagcagtcactgcaggcctcttggattgtacccgtaattacagcaattgccattagatttacgaagtcatttaattaaatggtttgatttatctccttaaatgcttccagaaatgggactagacttttttcactcaaacctgttcacaattattgctctttttcaattataaggtaaacaaggccatctatcagcaacacagtgctcgcattttttaattaaactatataaaaccaactatttgtggttgcgacttcactttttgttgaattactacccaatcattaatattgaagatgtgagatcatagatttattggctttgggcatctcaaatcccaagaggtcattttaaccaacaacattttaaaaagtagatttgtctgcctcagctatgagatgcgcatgtccctagcatctcatatctggttatattattttttccacttggtgaatgttgaaaaaaacaccactcgtccaatttatcagtttgcaggtctaatgtccttccctgttatttaaactgtatattgtaagcatgtcttatcgaaacaacttactcagttgtccgaaaacaaaactgcaaattctgtgtgtattcacgtactagaatcctgtcaaattggatcttgatttaagcttttatagcaacgaactttgcatactaagttttttttgttaaccggaactgccaagaagcattcagtaaaatacatcttcatcatttactgataatactcattcagactcatatcatactatttcgaattcattatacatcctcaaaa

SEQ ID NO: 45 - DNA, 725 bp, Saccharomyces cerevisiae
gctaggatctatatgcgaatatatcacatatgtaaattataagctcatcgcaaaaccaaaaaaaaaaaaattttcaataatttttcactaatcttcaaaaacaaatggggtaacccgtacaagagttattaaaacccaaaatgacaaaatcgcgacaattcaatcctacttaattagcaataacatactagcggtagagctactatcacatgttgaaccttgaatgctcaattcattgtactcaatactgctatcaaaagaaaaaaaatgtattaattatattcttgtcaaaatcaattttacactataagaggaaaatgttcttcagtcctagtaacattagttttctccctttgctagagactttacataatatcctagaaggtaaaattcgataatacagcagtaaagtcgtatattggtagcaatccttggtgacgctgactttttttttttgtaattttattgtttagttcatgataaaaaacttcaaatcacttttaatctggtagacagagaaaacaaatcgaaacgaaaatagagaactacgaataaaaaaatataagtggagaagatcgtcactacgcattaaacaatattgatcgctcaatgccagtactgcgcgtaaaagtttagtaacttaacgatttaggcacaatttgagaaaaatttcgccctgcagtaagtatgttattcagtacgatataaagctgaggttttatgct

SEQ ID NO: 46 - DNA, 881 bp, Saccharomyces cerevisiae
ggaagttgaagtttttcaaaaatttgctgtaaaatttgaccgatttgtgagattcttcctggctgtcagaatatggggccgtagtatattgtcagacctgttcctttaagaggtgatggtgataggcgttgagtatgtgtagtgtttgacccgagggtatggttttcacaagtactgcgcactgtattgtgaaagcagcttcggggtgcgtgattaaaaaatgcgaccaagaataaacaggtacatcataacaagggccatttgaattgcatttatcaggatttgtaaccttgttctaaagaggcatcgtatagtttaagttcattttccacccaatttgatgacggtgtggaccttaacctattgtcttgaaatttaggttatctcttagatatcacatgtgattaccccagtgaacgcgtataagcttacagaaaggaaaaccggttggctcagtcaaaactgttgcagatttgggctcccctgaatatttgagacatccctaaaatgaagagatatatacagctaattttgaatgaaaatttaaaattcgcaatgaacagtactagagatgagcttttgaagtcctttcaaattatttgttcttccagttgatattttttattttatataccagtaccaaatataatcttgccatacatttacctttttgaggttgttcaacggaaatccagtgtatttacacattcttggaaacccatcgcttataatacgaactaatttatttatgaacaaaggctttggaaaagtatccctactttttacgacgctaaatcatgatacgaaactttaggaagattaacagtcactccataaaatcagaaagtattcgctaatagtggaaagaaatggttatataa